(19) United States
(12) Patent Application Publication
(10) Pub. No.: US 2008/0176889 A1
(43) Pub. Date: Jul. 24, 2008

(54) 9-anilinoacridine alkylating agents

(76) Inventors: Tsann-Long Su, Taipei (TW); Ting-Chao Chou, Paramus, NJ (US)

Correspondence Address: FISH & RICHARDSON PC, P.O. BOX 1022, MINNEAPOLIS, MN, 55440-1022, US

(21) Appl. No.: 10/799,576

(22) Filed: Mar. 12, 2004

(51) Int. Cl. A61K 31/435; C07D 219/12; A61P 35/00

(52) U.S. Cl. 514/297; 546/106

(57) ABSTRACT

This invention relates to 9-anilinoacridine alkylating agents, their synthesis and their use in pharmaceutical compositions for treating diseases.

### TECHNICAL FIELD

This invention relates to 9-anilinoacridine alkylating agents, their synthesis and their use in pharmaceutical compositions for treating diseases.

### BACKGROUND

Alkylating nitrogen mustard derivatives are believed to exert their cytotoxic effects by interstrand cross-linking of DNA. Thus, the design and synthesis DNA-directed alkylators represents an approach to the development of new anticancer drugs.

### SUMMARY

In one aspect, this invention features compounds having formula (I):

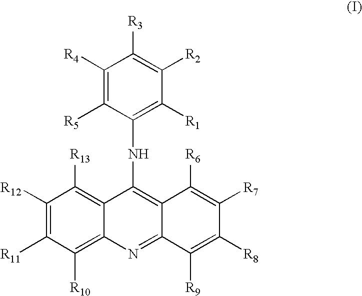

in which each of R₁, R₂, R₃, R₄, R₅, R₆, R₇, R₈, R₉, R₁₀, R₁₁, R₁₂, and R₁₃ is, independently, hydrogen, halo, nitro, hydroxyl, C₁-C₆ alkyl, C₁-C₆ alkoxy, C₁-C₆ hydroxyalkyl, CONHR^{a}, NR^{b}R^{c}, CONH(CH₂)ₘNR^{b}R^{c}, L-N(CH₂CH₂Cl)₂, or a DNA minor groove binder; L is (CH₂)ₚ or —O(CH₂)_{q}—; m is 1, 2, 3, or 4; p is 0, 1, 2, 3, or 4; q is 1, 2, 3, 4, 5, 6, 7, or 8; in which, R^{a} is C₁-C₆ alkyl; each of R^{b} and R^{c} is, independently, hydrogen, C₁-C₆ alkyl, COR^{d}, or COOR^{d}; R^{d} is C₁-C₆ alkyl C₆-C₁ aryl, or C₇-C₁₂ aralkyl; and provided that at least one of R₁, R₂, R₃, R₄, R₅, R₆, R₇, R₈, R₉, R₁₀, R₁₁, R₁₂, and R₁₃ is L-N(CH₂CH₂Cl)₂, or a salt thereof.

Embodiment can include one or more of the following features.

L can be (CH₂)ₚ, and p can be 0 or 1.

L can be —O(CH₂)_{q}—, and q can be 2 or 4.

One of R₁, R₂, R₃, R₄, or R₅ can be L-N(CH₂CH₂Cl)₂, e.g., one of R₂ or R₃ can be L-N(CH₂CH₂Cl)₂.

For example, R₂ can be L-N(CH₂CH₂Cl)₂, and L can be (CH₂)ₚ, and p can be 0 or 1 or L can be —O(CH₂)_{q}—, and q can be 2 or 4. Each of R₁, R₃, R₄, and R₅ can be, independently, hydrogen, C₁-C₆ alkyl, C₁-C₆ alkoxy, or C₁-C₆ hydroxyalkyl, e.g., R₄ can be C₁-C₆ hydroxyalkyl, e.g., CH₂OH, or R₁, R₃, R₄, and R₅ can all be hydrogen. As another example, R₃ can be L-N(CH₂CH₂Cl)₂, and L can be (CH₂)ₚ, and p can be 0 or 1, or L can be —O(CH₂)_{q}—, and q can be 2 or 4. Each of R₁, R₂, R₄, and R₅ can be, independently, hydrogen, C₁-C₆ alkyl, C₁-C₆ alkoxy, or C₁-C₆ hydroxyalkyl, e.g., each of R₁, R₂, R₄, and R₅ can be hydrogen.

Each of R₆, R₇, R₈, R₉, R₁₀, R₁₁, R₁₂, and R₁₃ can be, independently, hydrogen, halo, nitro, C₁-C₆ alkyl, C₁-C₆ alkoxy, CONHR^{a}, CONH(CH₂)ₘNR^{b}R^{c}, L-N(CH₂CH₂Cl)₂, or a DNA minor groove binder.

Each of R₆, R₇, R₈, R₉, R₁₀, R₁₁, R₁₂, and R₁₃ can be, independently, hydrogen, C₁-C₆ alkyl, CONH(CH₂)ₘNR^{b}R^{c}, L-N(CH₂CH₂Cl)₂, or a DNA minor groove binder.

One of R₉ and R₁₀ can be CONH(CH₂)ₘNR^{b}R^{c}, L-N(CH₂CH₂Cl)₂, or a DNA minor groove binder, and the other can be C₁-C₆ alkyl or hydrogen. For example, one of R₉ and R₁₀ can be CONH(CH₂)ₘNR^{b}R^{c} (e.g., CONH(CH₂)₂N(CH₃)₂), and the other can be C₁-C₆ alkyl (e.g., CH₃) or hydrogen. As another example, one of R₉ and R₁₀ can be L-N(CH₂CH₂Cl)₂ (e.g., one of R₉ and R₁₀ can be N(CH₂CH₂Cl)₂ or CH₂N(CH₂CH₂Cl)₂ or one of R₉ and R₁₀ can be O(CH₂)₂N(CH₂CH₂Cl)₂ or O(CH₂)₄N(CH₂CH₂Cl)₂), and the other can be C₁-C₆ alkyl (e.g., CH₃) or hydrogen. As a further example, one of R₉ and R₁₀ can be a DNA minor groove binder and the other can be C₁-C₆ alkyl (e.g., CH₃) or hydrogen. One of R₉ and R₁₀ can be CONH(CH₂)ᵣ-J-W—(CH₂)ₜR^{c}, in which r is 1, 2, 3, 4, or 5; t is 1, 2, 3, or 4, 5, or 6; J is —CONH— or —NHCO—; W is:

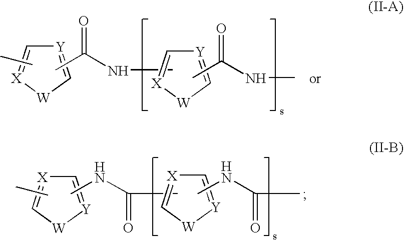

s is 0, 1, 2, 3, or 4; each of X and Y is, independently, N or CR^{f}; W is NR^{g}, O, or S; R^{e} is NR^{b}R^{c}, NHCHO, or NHC(═NH)NH₂; each of R^{b} and R^{c} is, independently, hydrogen, C₁-C₆ alkyl, COR^{d}, or COOR^{d}; and each of R^{f} and R^{g} is, independently, hydrogen or C₁-C₆ alkyl. s can be 0, each of X and Y can be CH, and W can be NCH₃. One of R₉ and R₁₀ can be:

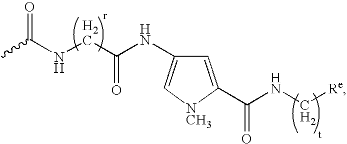

in which r and t can both be 3, and R^{e} can be N(CH₃)₂, NHCHO, or NHC(═NH)NH₂. One of R₉ and R₁₀ can be:

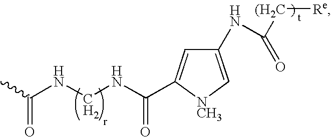

in which r and t can both be 3, and R^{e} can be N(CH₃)₂, NHCHO, or NHC(═NH)NH₂. In still another example, R₆, R₇, R₈, R₉, R₁₀, R₁₁, R₁₂, and R₁₃ each can be hydrogen.

One of R₆, R₇, R₈, R₉, R₁₀, R₁₁, R₁₂, and R₁₃ can be L-N(CH₂CH₂Cl)₂, e.g., R₉ can be L-N(CH₂CH₂Cl)₂, and L can be (CH₂)ₚ, and p can be 0 or 1 or L can be —O(CH₂)_{q}—, and q can be 2 or 4. Each of R₆, R₇, R₉, R₁₀, R₁₁, R₁₂, and R₁₃ can be, independently, hydrogen, halo, nitro, hydroxyl, C₁-C₆ alkyl, or C₁-C₆ alkoxy. Each of R₁, R₂, R₃, R₄, or R₅ is, independently, hydrogen, hydroxyl, C₁-C₆ alkyl, C₁-C₆ alkoxy, C₁-C₆ hydroxyalkyl, or NR^{b}R^{c}, e.g., R₂ can be hydroxyl or NR^{b}R^{c} (e.g., NH₂ or NHCOOCH₂CH₃) and R₄ can be C₁-C₆ hydroxyalkyl (e.g., CH₂OH).

In another aspect, the invention features a pharmaceutical composition that contains an effective amount of at least one of the 9-anilinoacridines described above, e.g., a compound having formula (I), and a pharmaceutically acceptable carrier. Also within the scope of this invention is a composition containing one or more of the 9-anilinoacridine compounds described above for use in treating cancer, and the use of such a composition for the manufacture of a medicament for the just-mentioned use.

In a further aspect, this invention features a method of treating a subject (e.g., a mammal including mice, rats, cows, sheep, pigs, rabbits, goats, and horses, monkeys, dogs, cats, and preferably humans) having cancer including administering to the subject an effective amount of a compound of formula (I). The cancer can be a human leukemia, sarcoma, osteosarcoma, lymphoma, melanoma, ovarian, skin, testicular, gastric, pancreatic, renal, breast, prostate colorectal, head and neck, brain, esophageal, bladder, adrenal cortical, lung, bronchus, endometrial, cervical or hepatic cancer. In certain embodiments the method can further include identifying a subject. Identifying a subject in need of such treatment can be in the judgment of a subject or a health care professional and can be subjective (e.g., opinion) or objective (e.g., measurable by a test or diagnostic method).

In one aspect, this invention also relates to a method of making a compound described herein. Alternatively, the method includes taking any one of the intermediate compounds described herein and reacting it with one or more chemical reagents in one or more steps to produce a compound described herein.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. The term “hydroxyalkyl” refer to an alkyl moiety in which an alkyl hydrogen atom is replaced by a hydroxyl group, e.g., a hydroxymethyl group. The term “alkoxy” refers to an —O-alkyl radical.

The term “aryl” refers to an aromatic monocyclic, bicyclic, or tricyclic hydrocarbon ring system. Any ring atom can be substituted. Examples of aryl moieties include, but are not limited to, phenyl, naphthyl, and anthracenyl.

The term “alkylene” refers to a divalent alkyl, e.g., —CH₂-(methylene), —CH₂CH₂-(ethylene), and —CH₂CH₂CH₂-(propylene).

Shown below are exemplary compounds, compounds 37-59, of this invention:

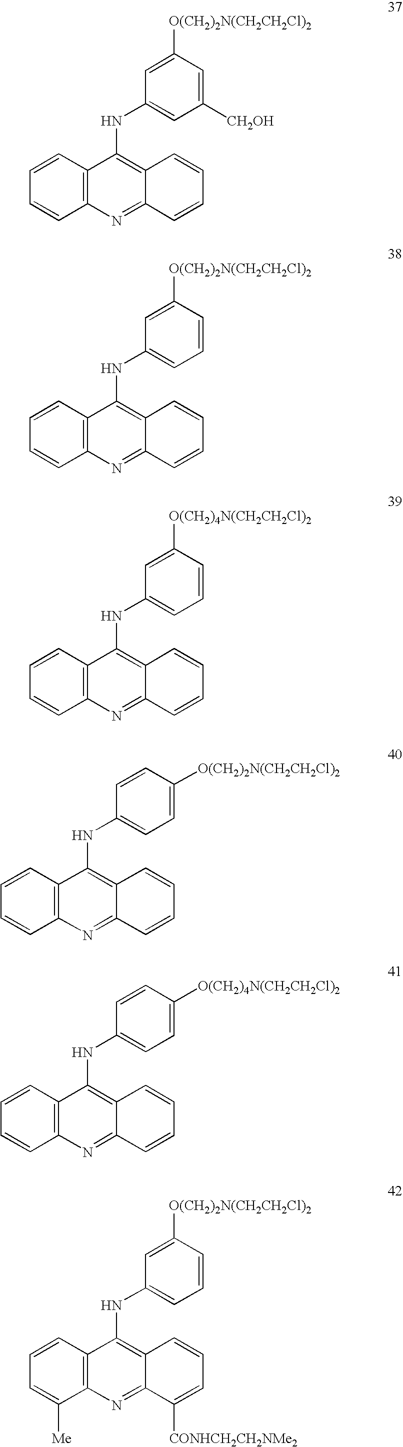

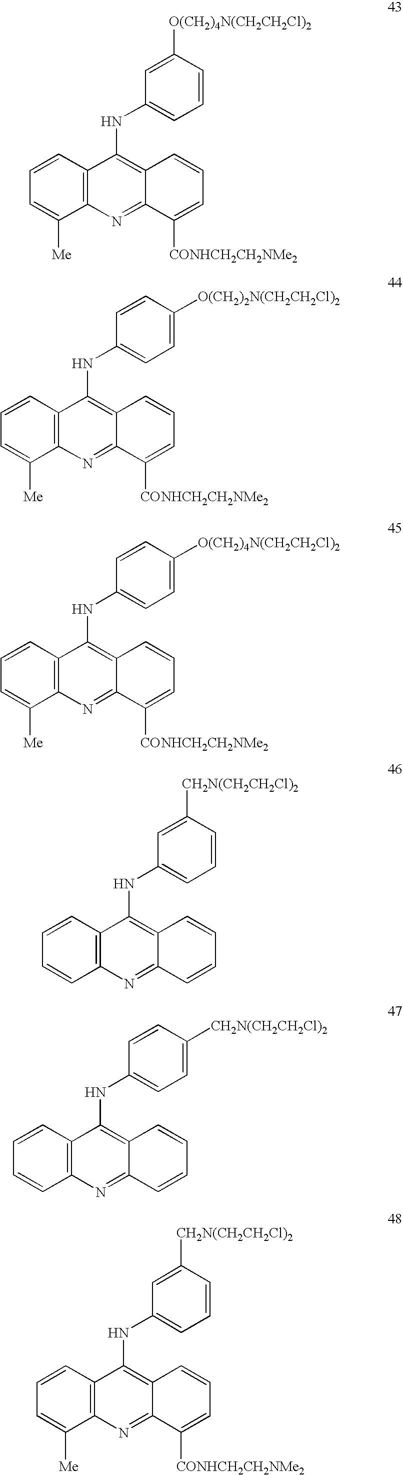

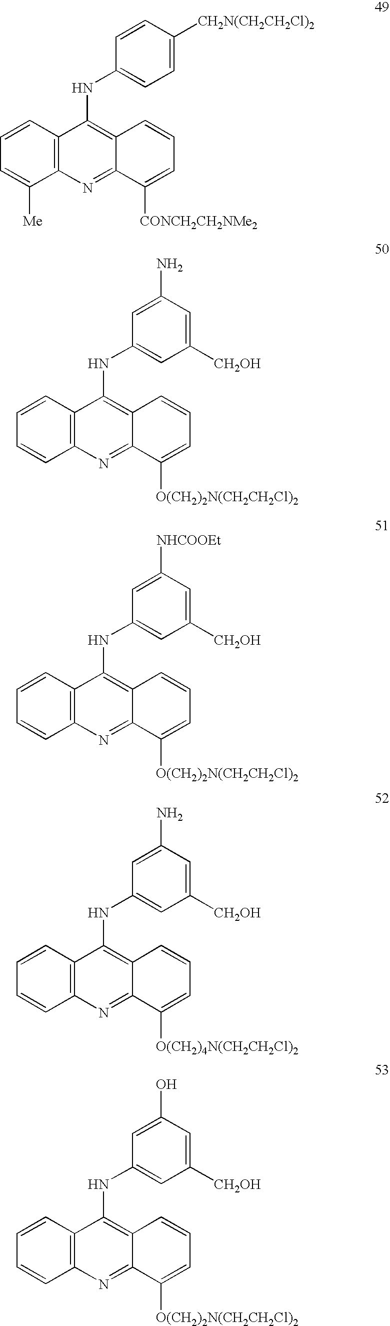

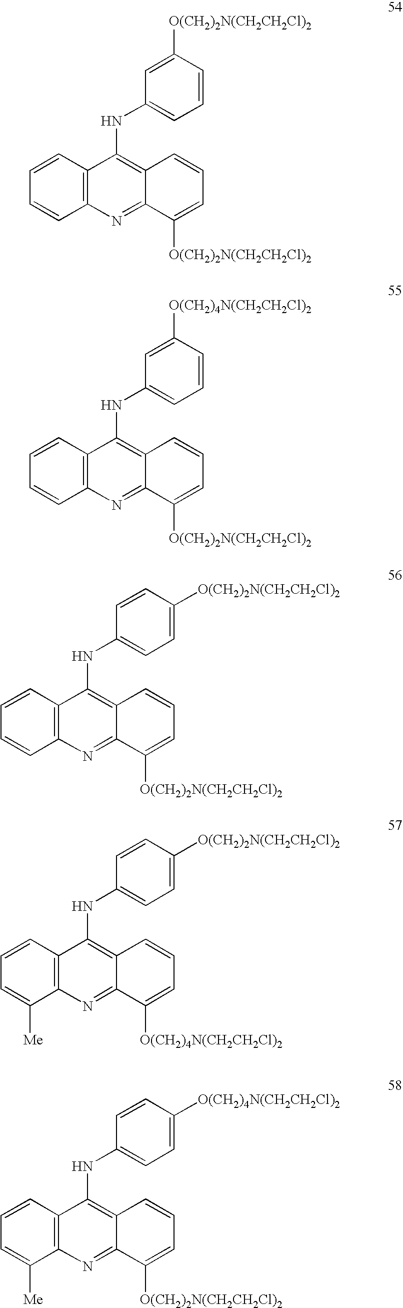

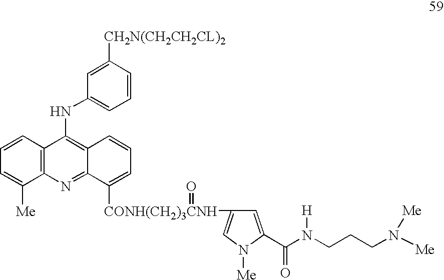

The 9-anilinoacridine compounds of this invention include the compounds themselves, as well as their salts and their prodrugs, if applicable. A salt, for example, can be formed between an anion and a positively charged substituent (e.g., amino or guanidinyl moieties) on a 9-anilinoacridine compound. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., phenolate) on a 9-anilinoacridine compound of this invention. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active 9-anilinoacridine compounds.

The details of one or more embodiments of the invention are set forth in the accompanying drawings and the description below. Other features and advantages of the invention will be apparent from the description and drawings, and from the claims.

### DETAILED DESCRIPTION

This invention relates in part to 9-anilinoacridine compounds of formula (I), which have one or more N-mustard alkylating moieties, e.g., -L-N(CH₂CH₂LG)₂, attached to one or more of the 9-anilinoacridine ring carbons. The term “L” represents an optional tether that links the nitrogen atom of the N-mustard alkylating moiety to one or more 9-anilinoacridine ring carbons. The tether can be alkylene, e.g., (CH₂)ₚ in which p is 1, 2, 3, or 4; or O-alkylene, e.g., O(CH₂)_{q} in which q is 1, 2, 3, 4, 5, 6, 7, or 8. In some embodiments, p is 1. In other embodiments, q is 2 or 4. When “L” is absent, the N-mustard alkylating moieties are attached to the 9-anilinoacridine ring carbons via the nitrogen atom, e.g., —N(CH₂CH₂LG)₂. Substituent “LG” in the formula -L-N(CH₂CH₂LG)₂ represents a leaving group, e.g., a chloro group.

The ring substituted with R₁-R₅ is referred to herein as the “aniline ring,” and the ring substituted with R₆-R₁₃ is referred to herein as the “acridine ring.”

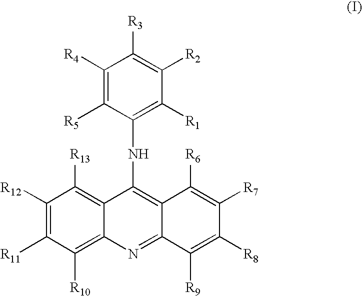

In some embodiments, one of the aniline ring substituents, R₁, R₂, R₃, R₄, or R₅, can be an N-mustard alkylating moiety, e.g., R₂ or R₃. In some embodiments, one of R₂ or R₃ can be N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂. The remaining four aniline ring substituents can be, independently of one another, hydrogen, C₁-C₆ alkyl (e.g., C₁ alkyl, C₂ alkyl, C₃ alkyl, C₄ alkyl, C₅ alkyl, or C₆ alkyl), C₁-C₃ alkoxy (e.g., C₁ alkoxy, C₂ alkoxy, or C₃ alkoxy), or C₁-C₃ hydroxyalkyl (e.g., C₁ hydroxyalkyl, C₂ hydroxyalkyl, or C₃ hydroxyalkyl). In some embodiments, when R₂ is N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, R₁, R₃, and R₅ can be hydrogen, and R₄ can be CH₂OH or hydrogen. In other embodiments, when R₃ is N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, R₁, R₃, R₄, and R₅ each can be hydrogen.

When the aniline ring is substituted with an N-mustard alkylating moiety, the acridine ring substituents, R₆, R₇, R₈, R₉, R₁₀, R₁₁, R₁₂, and R₁₃ can be, independently of one another, hydrogen, halo, nitro, C₁-C₆ alkyl, C₁-C₆ alkoxy, CONHR^{a}, CONH(CH₂)ₘNR^{b}R^{c}, L-N(CH₂CH₂LG)₂, or a DNA minor groove binder.

In one subset of compounds, R₆, R₇, R₈, R₉, R₁₀, R₁₁, R₁₂, and R₁₃ each can be hydrogen.

In another subset of compounds, one of R₆, R₇, R₈, R₉, R₁₀, R₁₁, R₁₂, or R₁₃ can be CONHR^{b} or CONH(CH₂)ₘNR^{b}R^{c}, and the remaining seven acridine substituents can be, independently of one another, hydrogen, C₁-C₆ alkyl (e.g., C₁ alkyl, C₂ alkyl, C₃ alkyl, C₄ alkyl, C₅ alkyl, or C₆ alkyl), or C₁-C₃ alkoxy (e.g., C₁ alkoxy, C₂ alkoxy, or C₃ alkoxy). In some embodiments, one of R⁹ and R¹⁰ can be CONH(CH₂)₂N(CH₃)₂ and the other can be C₁-C₆ alkyl (e.g., CH₃) or hydrogen. In certain embodiments, R₆-R₈ and R₁₁-R₁₃ can each be hydrogen.

In another subset of compounds, one of R₆, R₇, R₈, R₉, R₁₀, R₁₁, R₁₂, or R₁₃ can be L-N(CH₂CH₂LG)₂, and the remaining seven acridine substituents can be, independently of one another, hydrogen, C₁-C₆ alkyl (e.g., C₁ alkyl, C₂ alkyl, C₃ alkyl, C₄ alkyl, C₅ alkyl, or C₆ alkyl), C₁-C₃ alkoxy (e.g., C₁ alkoxy, C₂ alkoxy, or C₃ alkoxy), nitro, or halo. The N-mustard alkylating moiety on the aniline ring and the N-mustard alkylating moiety on the acridine ring can be the same moieties or different ones. In some embodiments, R₉ can be N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂; R₆-R₈ can be hydrogen; R₁₀-R₁₃ can be, independently of one another, hydrogen, C₁-C₆ alkyl, C₁-C₃ alkoxy, nitro, or halo. In some embodiments, one of R⁹ and R¹⁰ can be N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, and the other can be C₁-C₆ alkyl (e.g., CH₃) or hydrogen. In certain embodiments, R₆-R₈ and R₁₁-R₁₃ can each be hydrogen.

In another subset of compounds, one of R₆, R₇, R₈, R₉, R₁₀, R₁₁, R₁₂, or R₁₃ can be a DNA minor groove binder, e.g., netropsin or distamycin analogues, and the remaining seven acridine substituents can be, independently of one another, hydrogen, C₁-C₆ alkyl (e.g., C₁ alkyl, C₂ alkyl, C₃ alkyl, C₄ alkyl, C₅ alkyl, or C₆ alkyl), C₁-C₃ alkoxy (e.g., C₁ alkoxy, C₂ alkoxy, or C₃ alkoxy), nitro, or halo.

In general, the DNA minor groove binder can have the formula, —CONH(CH₂)ᵣ-J-W—(CH₂)ₜR^{e}, in which the amide carbonyl carbon at the left hand side of the formula represents the point of attachment of the DNA minor groove binder to the acridine ring. The spacers “(CH₂)ᵣ” and “(CH₂)ₜ” can each contain, independently of one another, 1-5 CH₂ units (e.g., 1, 2, 3, 4, or 5 CH₂ units) and 1-6 CH₂ units (e.g., 1, 2, 3, 4, 5, or 6 CH₂ units), respectively. In certain embodiments, both r and t are 3. The term “J” can either be —CONH— or —NHCO—. The term “W” represents a heteroaryl group having either formula (II-A) or (II-B) shown below. W can be a monomeric, dimeric, trimeric, tetrameric, or pentameric entity, i.e., s can be 0, 1, 2, 3, or 4, respectively. Any ring atom capable of being substituted can be the point of attachment for the intervening amide linkages shown in formulas (II-A) and (II-B). Each of the five membered rings can

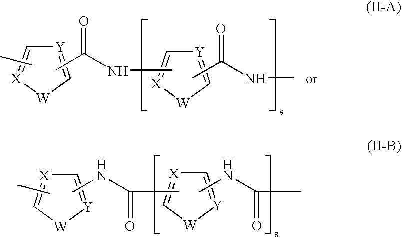

contain 1, 2, or 3 heteroatoms. In some embodiments, W can be NR^{g}, O, or S; and X and Y can be, independently of one another, N or CR^{f}, in which R^{f} and R^{g} can either be hydrogen or C₁-C₆ alkyl (e.g., C₁ alkyl, C₂ alkyl, C₃ alkyl, C₄ alkyl, C₅ alkyl, or C₆ alkyl). In some embodiments, W is NCH₃; and X and Y can both be CH; or X can be CH and Y can be N; or X can be N and Y can be CH; or X and Y can both be N. R^{e} can be NR^{b}R^{c}, NHCHO, or NHC(═NH)NH₂. Each of R^{b} and R^{c} can be, independently of one another, hydrogen, C₁-C₆ alkyl (e.g., C₁ alkyl, C₂ alkyl, C₃ alkyl, C₄ alkyl, C₅ alkyl, or C₆ alkyl), COR^{d}, or COOR^{d}, in which R^{d} can be C₁-C₆ alkyl (e.g., C₁ alkyl, C₂ alkyl, C₃ alkyl, C₄ alkyl, C₅ alkyl, or C₆ alkyl), C₆-C₁₀ aryl (e.g., phenyl) or C₇-C₁₂ aralkyl (e.g., benzyl). In some embodiments, R^{e} can be N(CH₃)₂, NHCHO, or NHC(═NH)NH₂ (or the acid salts thereof). In some embodiments, the DNA minor groove binder can have the structure represented by formula (III) or (IV).

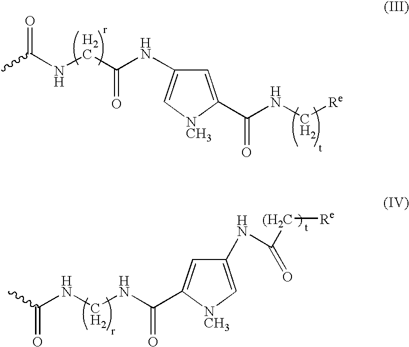

In some embodiments, R₉ can be a DNA minor groove binder having formula (III) or (IV). R₆-R₈ can be hydrogen, and R₁₀-R₁₃ can be, independently of one another, hydrogen, C₁-C₆ alkyl, C₁-C₃ alkoxy, nitro, or halo. In some embodiments, one of R⁹ and R¹⁰ can be a DNA minor groove binder having formula (III) or (IV), and the other can be C₁-C₆ alkyl (e.g., CH₃) or hydrogen. In some embodiments, r and t are both 3, and R^{e} is N(CH₃)₂, NHCHO, or NHC(═NH)NH₂ (or the acid salts thereof). In certain embodiments, R₆-R₈ and R₁₁-R₁₃ can each be hydrogen.

In some embodiments, one of the acridine ring substituents, R₆, R₇, R₈, R₉, R₁₀, R₁₁, R₁₂, or R₁₃, can be an N-mustard alkylating moiety, and each of the aniline ring substituents, R₁, R₂, R₃, R₄, or R₅, can be, independently of one another, hydrogen, hydroxyl, C₁-C₆ alkyl, C₁-C₆ alkoxy, C₁-C₆ hydroxyalkyl, or NR^{b}R^{c}.

In certain embodiments, R₉ can be N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, and the remaining seven acridine substituents can be, independently of one another, hydrogen, C₁-C₆ alkyl (e.g., C₁ alkyl, C₂ alkyl, C₃ alkyl, C₄ alkyl, C₅ alkyl, or C₆ alkyl), C₁-C₃ alkoxy (e.g., C₁ alkoxy, C₂ alkoxy, or C₃ alkoxy), nitro, or halo. In some embodiments, when R₉ is N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, R₆-R₈ can be hydrogen, and each of R₁₀-R₁₃ can be, independently of one another, hydrogen, C₁-C₆ alkyl, C₁-C₃ alkoxy, nitro, or halo. In some embodiments, one of R⁹ and R¹⁰ can be N(CH₂CH₂Cl)₂, CH₂N(CH₂CH₂Cl)₂, O(CH₂)₂N(CH₂CH₂Cl)₂ or O(CH₂)₄N(CH₂CH₂Cl)₂, and the other can be C₁-C₆ alkyl (e.g., CH₃) or hydrogen. In certain embodiments, R₆-R₈ and R₁₁-R₁₃ can each be hydrogen.

In certain embodiments, R₂ can be hydroxyl or NR^{b}R^{c}, and R₄ can be C₁-C₆ hydroxyalkyl (e.g., C₁ hydroxyalkyl, C₂ hydroxyalkyl, or C₃ hydroxyalkyl). Each of R^{b} and R^{c} can be, independently of one another, hydrogen, C₁-C₆ alkyl (e.g., C₁ alkyl, C₂ alkyl, C₃ alkyl, C₄ alkyl, C₅ alkyl, or C₆ alkyl), COR^{d}, or COOR^{d}, in which R^{d} can be C₁-C₆ alkyl (e.g., C₁ alkyl, C₂ alkyl, C₃ alkyl, C₄ alkyl, C₅ alkyl, or C₆ alkyl), C₆-C₁₀ aryl (e.g., phenyl) or C₇-C₁₂ aralkyl (e.g., benzyl). In some embodiments, R₂ can be NH₂ or NHCOOCH₂CH₃, and R₄ can be CH₂OH.

The compounds of this invention can be synthesized using conventional techniques. Advantageously, these compounds are conveniently synthesized from readily available starting materials. In general, the compounds of the formulae described herein are conveniently obtained via standard organic chemistry synthesis methods, including those methods illustrated in the schemes and the examples herein.

An exemplary scheme of synthesizing the 9-anilinoacridines of this invention is presented below (for definitions of R₁-R₁₃, see Formula I). In some embodiments, any one of R₁-R₁₃ can be, for example, a synthetic precursor or protected form of the substituents corresponding to R₁-R₁₃. 9-Acridone A can be converted to compound B, which contains a leaving group “L” at the 9-position. The leaving group may be halo, triflate, mesylate, nosylate or phenoxy. Preferably, L is chloro, e.g., via reaction of 9-acridones with a chlorinating agent, e.g., thionyl chloride. Anilinoacridines having formula (I) can be obtained via the condensation of aniline C with B, e.g., by nucleophilic displacement of the leaving group L in B with the amino group in C.

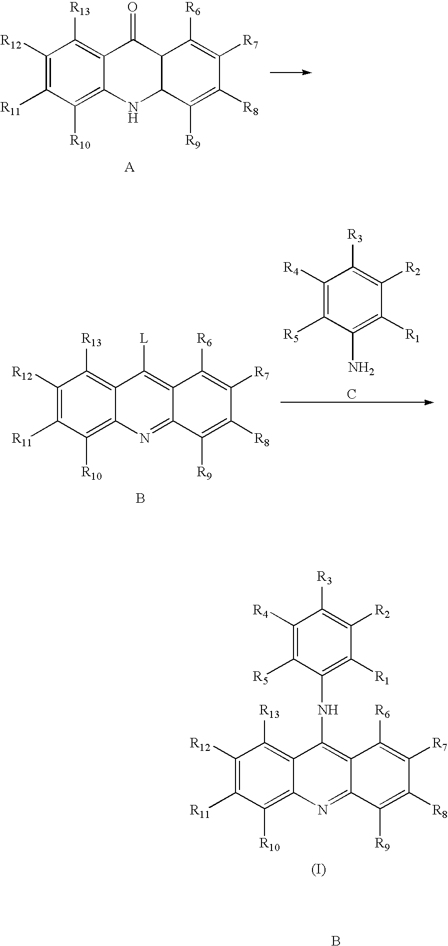

Nucleophilic agents are known in the art and are described in the chemical texts and treatises referred to herein, and include reagents having electrons to share. Leaving groups are known in the art and are any stable species that can be detached from a molecule during a reaction (e.g., halides, triflates, mesylate, nosylate, phenoxy alkoxides, alkylmercapto or amino). The chemicals used in the aforementioned methods can include, for example, solvents, reagents, catalysts, protecting group and deprotecting group reagents and the like. The methods described above can also additionally include steps, either before or after the steps described specifically herein, to add or remove suitable protecting groups in order to ultimately allow synthesis of the compound of the formulae described herein.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

To illustrate, the syntheses of various exemplary compound subsets are delineated in the schemes below.

For example, condensation of an aminophenol, e.g., 7, with a 9-chloroacridine, e.g., 8, can provide (9-acridinylamino)phenol derivatives, e.g., 9, which, in turn can be treated with, e.g., tris(2-chloroethyl)amine to give compounds having formula 10 (see Scheme 1 below).

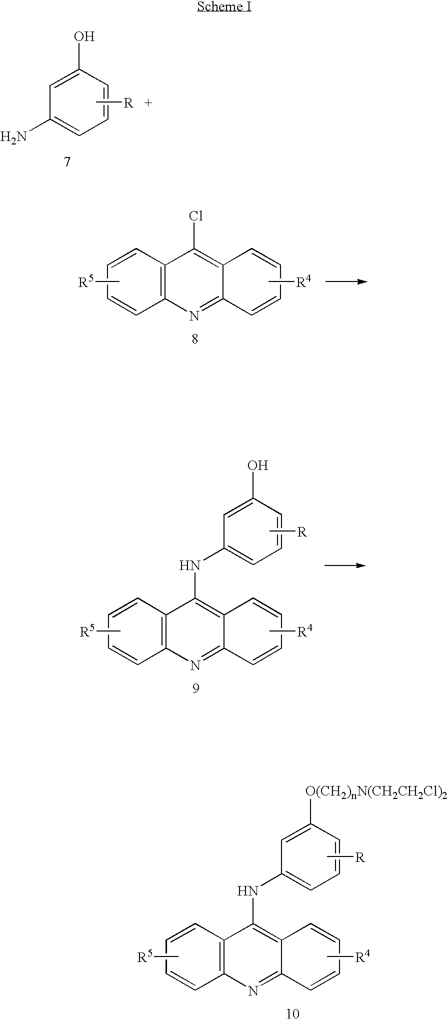

In some embodiments, one or more N-mustard alkylating moieties can be introduced onto the aniline and/or acridine condensation partners, e.g., B and/or C, respectively, prior to formation of the anilinoacridine compounds.

For example, reaction of a nitrophenol, e.g., 11, with, e.g., an α,ω-dibromoalkanes (such as 1,4-dibromobutane), can can afford monohalo compounds, e.g., 12, which, in turn, can then be treated with excess diethanolamine to afford bis(ethanolamino) compounds, e.g., 13. Chlorination of the bis(ethanolamino) compounds (e.g., using methanesulfonyl chloride/triethylamine in dichloromethane at about 0° C.) can provide compounds having structure 14. The nitro group in, e.g., 14, can be reduced to an amino group (e.g., using stannic chloride in concentrated hydrochloric acid) to yield anilines, e.g., 15, having an N-mustard alkylating moiety (see Scheme 2 below). Condensation of anilines, e.g., 15, with 9-chloroacridines, e.g., 8, can give compounds having structure 16.

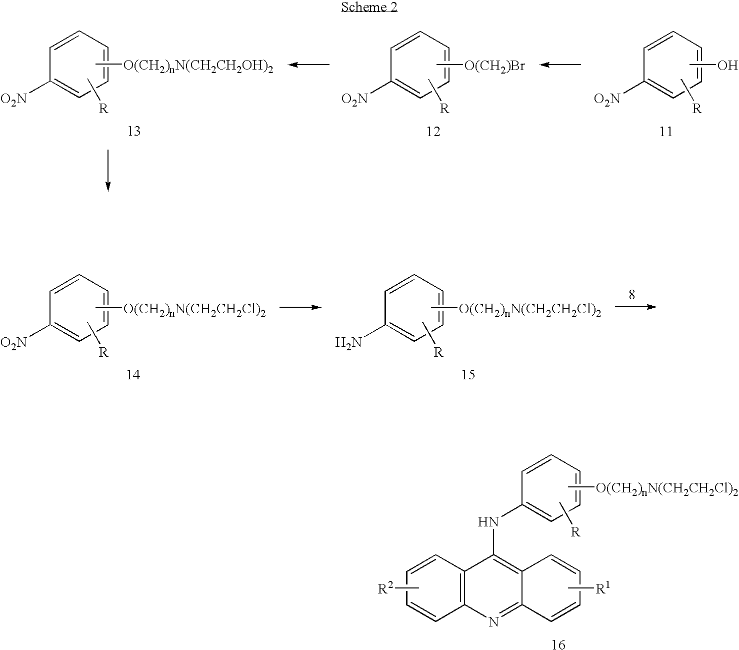

Similarly, compounds having structure 20 can be prepared, for example, from nitrobenzyl bromides, e.g., 17 (e.g., via nucleophilic displacement with diethanolamine to form, e.g., 18; followed by chlorination with methanesulfonyl chloride/pyridine to form, e.g., 19; and followed by reduction with SnCl₂/HCl to form, e.g., 20). These steps are delineated in Scheme 3 below. Again, condensation of anilines, e.g., 20, with 9-chloroacridines, e.g., 8 can give compounds having structure 21.

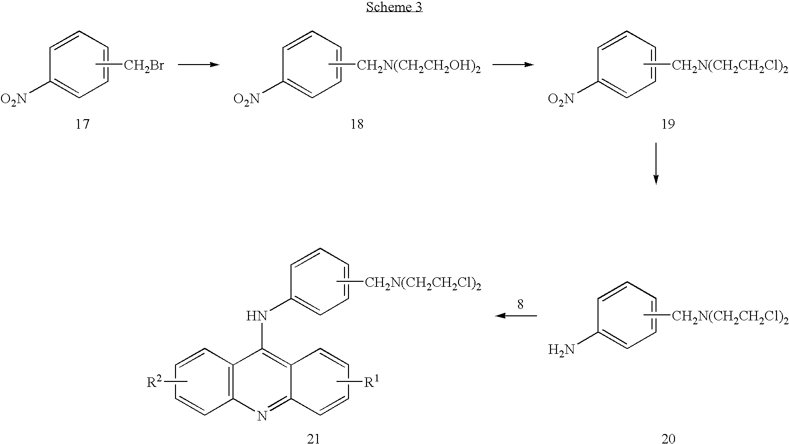

In some embodiments, 4-hydroxyacridin-9-ones, e.g., 22, can be used to form acridin-9-ones having one or more N-mustard alkylating moieties attached to the acridine ring, e.g., 25 (e.g., via reaction with, α,ω-dibromoalkanes (such as 1,4-dibromobutane) to form, e.g., 23; followed by nucleophilic displacement with diethanolamine to form, e.g., 24; and followed by chlorination with methanesulfonyl chloride/pyridine to form, e.g., 25). Acridin-9-ones having N-mustard alkylating moieties, e.g., 25, can be converted to 9-chloroacridine compounds, e.g., 26, which, in turn, can be condensed with anilines, e.g., 27, to provide 9-anilinoacridines having structure 28. These steps are delineated in Scheme 4 below.

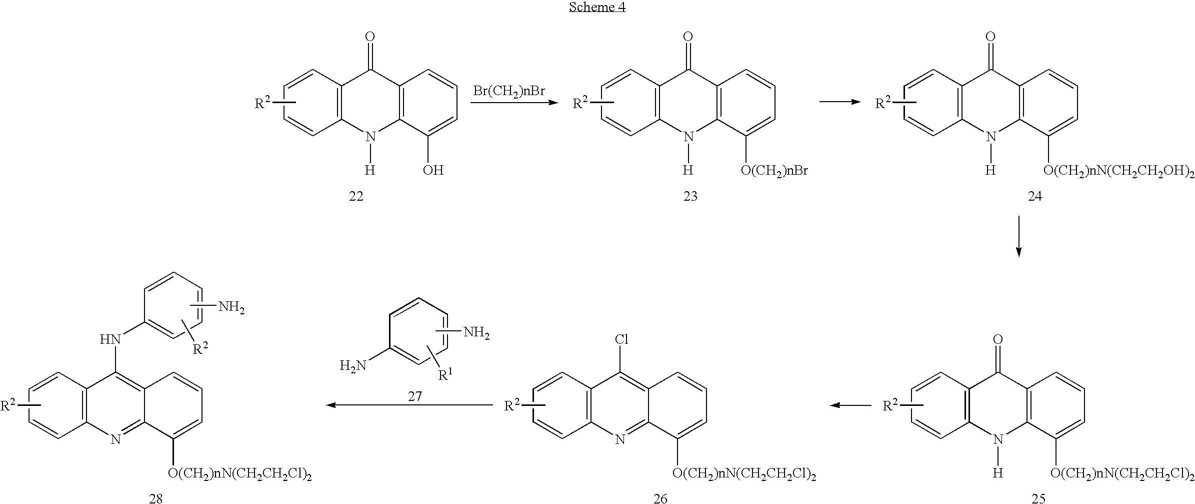

Compounds having N-mustard alkylating moieties on both the aniline and acridine rings can be prepared, e.g., by the condensation of anilines, e.g., 15 or 20, with 9-chloroacridines, e.g., 26 (see Scheme 5 below).

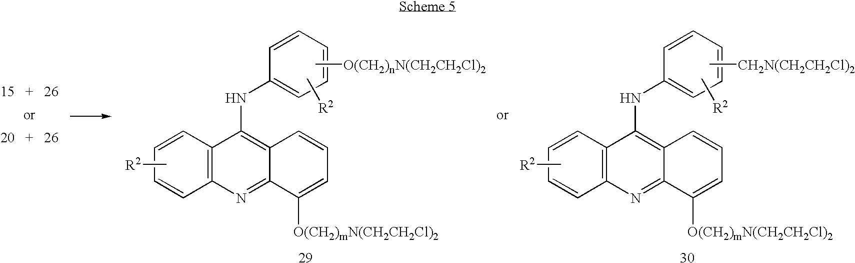

Compounds having N-mustard alkylating moieties on the aniline ring and either DNA minor groove binders or CONH(CH₂)ₘNR^{b}R^{c} on the acridine ring can be prepared as follows. Exposure of a 9-oxoacridan-4-carboxylic acid, e.g., 31, to a chlorinating agent, e.g., thionyl chloride (and optionally a catalytic amount of dimethylformamide (DMF)) can result in the conversion of both the 9-oxo and 4-carboxy groups to the corresponding 9-chloro and 4-acid chloride groups, respectively, resulting in dichloro compounds, e.g., 32 (see Scheme 6 below). In some embodiments, it may be desirable not to isolate the dichlorocompounds before using them in subsequent synthetic transformations. In some embodiments, a dichloro compound can be combined first with an aliphatic amine, e.g., N,N-dimethylethylenediamine, and then with an aniline having one or more N-mustard alkylating moieties, e.g., 15 or 20, to provide 9-anilinoacridine compounds such as 42-45. Similarly, a dichloro compound, e.g., 32, can be combined first with a DNA minor groove binder, e.g., 33, and then with an aniline having one or more N-mustard alkylating moieties, e.g., 15 and 20, to provide compounds having structures 35 or 36 (see Scheme 6 below).

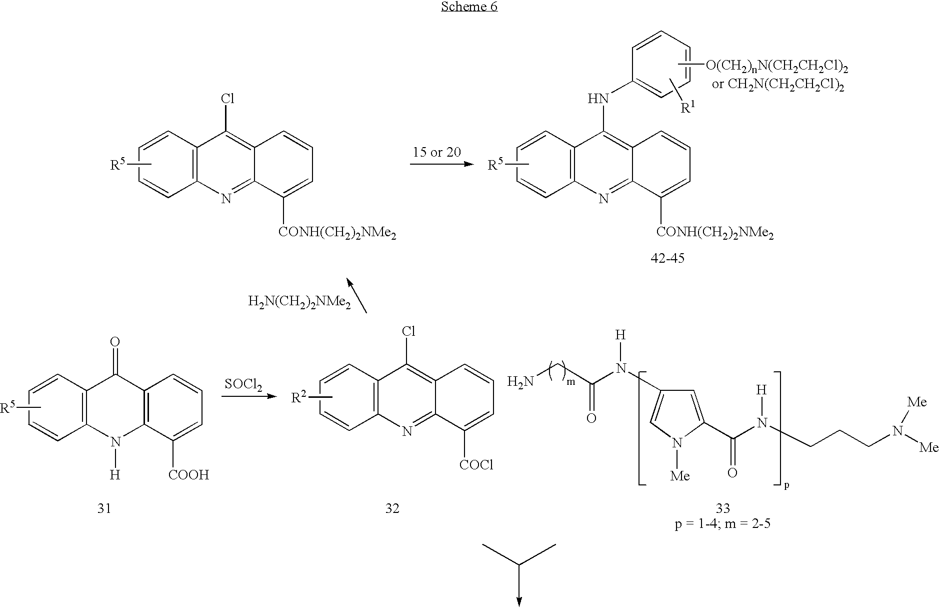

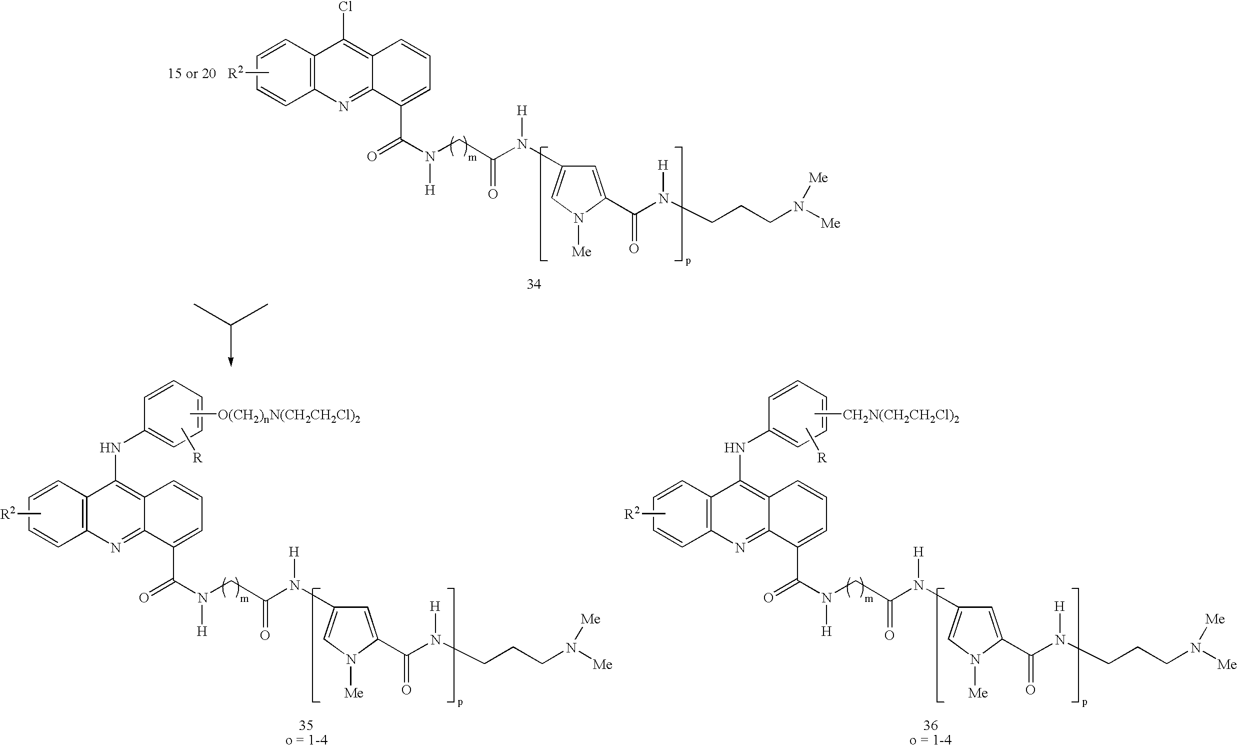

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The compounds of this invention may also contain linkages (e.g., carbon-carbon bonds; carbon-nitrogen bonds, e.g., amides) wherein bond rotation is restricted about that particular linkage, e.g. restriction resulting from the presence of a ring or double bond. Accordingly, all cis/trans and E/Z isomers, and rotamers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

It is understood that the actual electronic structure of some chemical entities cannot be adequately represented by only one canonical form (e.g., Lewis structures). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

Also within the scope of this invention is a pharmaceutical composition that contains an effective amount of at least one 9-anilinoacridine compound of this invention and a pharmaceutically acceptable carrier. Further, this invention covers a method of administering an effective amount of one or more of such 9-anilinoacridine compounds to a cancer patient. “An effective amount” refers to the amount of an active 9-anilinoacridine compound that is required to confer a therapeutic effect on the treated subject. An effective amount may range from about 0.1 mg/Kg to about 500 mg/Kg, e.g., 1 mg/Kg to about 50 mg/Kg. Effective doses will vary, as recognized by those skilled in the art, depending on the types of diseases treated, route of administration, excipient usage, and the possibility of co-usage with other therapeutic treatment. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

“Treating” refers to administering a compound described herein to a subject that prevents, cures, heals, alleviates, relieves, alters, remedies or ameliorates any primary phenomena (e.g., initiation, progression, metastasis) and/or secondary symptoms associated with the diseases delineated herein

To practice the method of the present invention, a composition having one or more 9-anilinoacridne compounds can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active 9-anilinoacridine compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active indolizine compound. Examples of other carriers include colloidal silicon oxide, magnesium stearate, cellulose, sodium lauryl sulfate, and D&C Yellow # 10.

The 9-anilinoacridines compounds of this invention can be preliminarily screened for their efficacy in treating cancers by one or more of the following in vitro assays and in vivo assays discussed below. Other methods will also apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

The synthesis of 4-hydroxy-10H-acridin-9-one is described in, e.g., Su, T.-L., et al., J. Med. Chem. 1995, 38, 3226-3235. The synthesis of 3-amino-5-hydroxymethylphenol is described in, e.g., Su, T.-L., et al., J. Med. Chem. 1999, 42, 4741-4748. The synthesis of 9-chloro-5-methylacridan-4-dimethylaminoethylcarboxamide is described in, e.g., Su, T.-L., Current Med. Chem. 2002, 9, 1677-1688.

### Example 1

### 4-{2-[Bis-(2-chloroethyl)amino]ethoxy}-10H-acridine-9-one (60)

A mixture of tris(2-chloroethyl)amine hydrochloride (9.64 g, 40 mmol) and dry powdered K₂CO₃ (6.91 g, 50 mmol) in dry DMSO (15 mL) was stirred at room temperature for 1 h. A solution of 4-hydroxy-10H-acridin-9-one (2.12 g, 10 mmol) in dry DMSO (5 mL) was added into the above mixture and stirred at room temperature for 20 h. The reaction mixture was poured onto ice water (100 mL) and extracted with EtOAc (100 mL×5). The organic extracts were combined, washed with ice water, dried over Na₂SO₄ and evaporated in vacuo to dryness. The residue was recrystallized from EtOH to give 60, 1.02 g (27%); mp 131-133° C.; ¹H NMR (DMSO-d₆) δ 3.01 (4H, t, J=6.74 Hz, 2×NCH₂), 3.20 (2H, t, J=5.66 Hz, NCH₂), 3.64 (4H, t, J=6.74 Hz, 2×CH₂Cl), 4.31 (2H, t, J=5.66 Hz, OCH₂), 7.18 (1H, m, ArH), 7.27 (1H, m, ArH), 7.49 (1H, m, ArH), 7.72 (1H, m, ArH), 7.82 (1H, m, ArH), 7.92 (1H, m, ArH), 8.23 (1H, m, ArH), 10.78 (1H, brs, exchangeable, NH); Anal. Calcd. for C₁₉H₂₀Cl₂N₂O₂; C, 60.16; H, 5.32; N, 7.39. Found. C, 60.13; H, 5.38; N, 7.35.

### Example 2

### 4-(4-bromobutoxy)-10H-acridin-9-one (61)

A solution of 4-hydroxy-10H-acridin-9-one (5.01 g, 24 mmol) and K₂CO₃ (6.64 g, 48 mmol) in DMF (35 mL) was stirred at room temperature for 5 min. 1,4-Dibromobutane (15.56 g, 72 mmol) was added to the above mixture and then stirred at 40° C. for 2 h. The reaction mixture was filtered through a pad of Celite, washed with DMF. The combined filtrate and washings were evaporated in vacuo to remove DMF. The residue was diluted with water (30 mL) and extracted with CHCl₃ (50 mL×6). The organic extracts were combined, washed successively with 1% NaOH (50 mL) and water (30 mL), dried over Na₂SO₄, and evaporated under reduced pressure to dryness. The residue was chromatographed on a silica gel column (2×20 cm) using CHCl₃ as an eluent. The main fractions containing the desired product was collected, evaporated in vacuo to dryness and the residue was recrystallized from EtOH to give 61, 4.09 g (49.2%); mp 180-181° C.; ¹H NMR (DMSO-d₆) δ 2.15 (4H, m, CH₂CH₂), 3.60 (2H, t, J=6.06 Hz, CH₂), 4.24 (2H, t, J=8.96 Hz, CH₂), 7.06 (1H, m, ArH), 7.15 (1H, m, ArH), 7.41 (1H, m, ArH), 7.65 (1H, ArH), 8.04 (1H, m, ArH), 8.48 (1H, m, ArH). Anal. Calcd. for C₁₇H₁₆BrNO₂: C, 58.97; H, 4.66; N, 4.05. Found. C, 58.72; H, 4.63; N, 3.98.

### Example 3

### 4-{4-[Bis-(2-hydroxyethyl)amino]butoxy}-10H-acridin-9-one (62)

A mixture of 4-(4-bromobutoxy)-10H-acridin-9-one (61) (2.77 g, 8.0 mmol) and diethanolamine (5.27 g, 50 mmol) in diglyme (10 mL) was heated at 115° C. with vigorous stirring for 30 min. After cooling, the mixture was concentrated under reduced pressure to 5 mL. The oil syrup was triturated successively with hexane (50 mL×5) and ether (30 mL×2) and then dissolved with CHCl₃ (200 mL). The CHCl₃ solution was washed with water (80 mL×6) to remove excess diethanolamine, dried over Na₂SO₄, and evaporated in vacuo to dryness. The residue was crystallized from EtOH/hexane to give needle pale yellow crystals, 2.356 g (79.6%); mp 124-126° C.; ¹H NMR (DMSO-d₆) δ 1.64 (2H, m, J=9.27 Hz, CH₂), 1.99 (2H, m, J=10.8 Hz, CH₂), 2.59 (2H, t, J=5.76 Hz, OCH₂), 2.74 (4H, t, J=5.04 Hz, 2×CH₂OH), 3.76 (6H, m, J=6.37 Hz, 3×NCH₂), 6.62 (1H, m, ArH), 6.90 (1H, m, ArH), 7.15 (1H, m, ArH), 7.31 (1H, m, ArH), 7.44 (1H, m, ArH), 7.92 (1H, m, ArH), 8.40 (1H, m, ArH), 9.45 (1H, s, exchangeable, NH). Anal. Calcd. for C₂₁H₂₆N₂O₄: C, 68.07; H, 7.07; N, 7.56. Found. C, 67.82; H, 7.06; N, 7.48.

### Example 4

### 4-{4-[Bis-(2-chloroethyl)amino]butoxy}-10H-acridin-9-one (63)

Methanesulfonyl chloride (8.88 g, 75 mmol) was added dropwise to a solution of 4-{4-[bis-(2-hydroxyethyl)amino]butoxy}-10H-acridin-9-one (62) (11.14 g, 30 mmol) and triethylamine (9.08 g, 90 mmol) in dry CHCl₃ (25 mL) in an ice-bath. The reaction mixture was stirred for 3 days at room temperature. The reaction mixture was diluted with CHCl₃ (150 mL), washed successively with water (50 mL×2), cold aqueous solution of NaHCO₃ (50 mL) and ice water (100 mL), dried over Na₂SO₄ and evaporated in vacuo to dryness. The residue was recrystallized from EtOH to give pale yellow crystal, 10.23 g (83.7%); mp 119-119° C.; ¹H NMR (DMSO-d₆) δ 1.64 (2H, brs, CH₂), 1.93 (2H, m, CH₂), 2.84 (2H, brs, NCH₂); 3.34 (4H, brs, 2×NCH₂), 3.61 (4H; brs, 2×CH₂Cl), 4.28 (2H, t, J=8.48 Hz, OCH₂), 7.18 (1H, m, ArH), 7.27 (1H, m, ArH), 7.34 (1H, m, ArH), 7.72 (1H, m, ArH), 7.80 (1H, m, ArH), 7.99 (1H, m, ArH), 8.23 (1H, m, ArH), 10.90 (1H, s, exchangeable, NH). Anal. Calcd. for C₁₉H₂₀Cl₂N₂O₂: C, 61.92; H, 5.94; N, 6.88. Found. C, 61.78; H, 5.92; N, 6.81.

### Example 5

### Bis-(2-Chloroethyl)-[2-(3-nitrophenoxy)ethyl]amine (64)

A mixture of m-nitrophenol (4.0 g, 28.75 mmol), tris(2-chloroethyl)amine hydrochloride (8.34 g, 34.5 mmol), KF (1.66 g, 28.75 mmol) and K₂CO₃ (19.84 g, 143.8 mmol) in dry acetone (200 mL) was heated at reflux for 2 days. After cooling, the reaction mixture was filtered, washed with acetone. The combined filtrate and washings were evaporated in vacuo to dryness, and the residue was dissolved in CHCl₃ (200 mL), washed with water (150 mL×3), dried over Na₂SO₄, and evaporated in vacuo to dryness. The residue was chromatographed on a silica gel column (5×7 cm) using CH₂Cl₂ as the eluent. The fractions containing the desired product were combined and evaporated under reduced pressure to give 64 as syrup, 2.2 g (25%); HCl salt; mp 119-120° C. (EtOH); ¹H NMR (CDCl₃) δ 3.05 (4H, t, J=6.91 Hz, 2×NCH₂), 3.10 (2H, t, J=5.53 Hz, NCH₂), 3.57 (4H, t, J=6.91 Hz, 2×CH₂Cl), 4.12 (2H, t, J=5.53 Hz, OCH₂), 7.21 (1H, dq, J=2.50, J=8.31 Hz, ArH), 7.41 (1H, t, J=8.81 Hz, ArH), 7.69 (1H, t, J=2.50 Hz, ArH), 7.79 (1H, dq, J=2.50, J=8.31 Hz, ArH). Anal. Calcd. for C₁₂H₁₆Cl₂N₂O₃·2HCl·2H₂O: C, 34.45; H, 5.30; N, 6.69. Found. C, 34.45; H, 5.31; N, 6.53.

### Example 6

### Bis-(2-Chloroethyl)-[2-(4-nitrophenoxy)ethyl]amine (65)

By following the same procedure as that for the synthesis of 64, bis-(2-chloroethyl)-[2-(4-nitrophenoxy)ethyl]amine (65) was prepared from p-nitrophenol (11.13 g, 80.0 mmol) and tris(2-chloroethyl)amine hydrochloride (21.2 g, 88 mmol): yield 5.3 g (21%) as syrup; HCl salt; mp 188-9° C. (EtOH); ¹H NMR (CDCl₃) δ 3.04 (4H, t, J=6.86 Hz, 2×NCH₂), 3.10 (2H, t, J=5.56 Hz, NCH₂), 3.55 (4H, t, J=6.86 Hz, 2×CH₂Cl), 4.13 (2H, t, J=5.56 Hz, OCH₂), 6.96 (2H, d, J=9.19 Hz, 2×ArH), 8.20 (2H, d, J=9.19 Hz, 2×ArH). Anal. Calcd. for C₁₂H₁₆Cl₂N₂O₃·HCl: C, 41.94; H, 4.99; N, 8.15. Found. C, 41.78; H, 5.04; N, 8.02.

By following the same procedure as that for the synthesis of 4-(4-bromobutoxy)-acridin-9-one (61), compounds 66 and 67 were prepared:

### Example 7

### 1-(4-Bromobutoxy)-3-nitrobenzene (66)

1-(4-Bromobutoxy)-3-nitrobenzene (66) was prepared from m-nitrophenol (4.99 g, 35.9 mmol) and 1,4-dibromobutane (2.81 g, 13.0 mmol): yield 2.58 g (62%) as syrup; ¹H NMR (CDCl₃-d) δ 2.02 (2H, m, CH₂), 2.08 (2H, m, CH₂), 3.50 (2H, t, J=6.20 Hz, CH₂Br), 4.78 (2H, t, J=6.20 Hz, OCH₂), 7.22 (1H, dq, J=2.69, J=8.31 Hz, ArH), 7.43 (1H, t, J=8.07 Hz, ArH), 7.69 (1H, dq, J=2.69, J=8.31 Hz, ArH), 7.89 (1H, s, exchangeable, NH). Anal. Calcd. for C₁₀H₁₂NO₃·0.1H₂O: C, 43.53; H, 4.46; N, 5.07. Found. C, 44.10; H, 4.52; N, 5.09.

### Example 8

### 1-(4-Bromobutoxy)-4-nitrobenzene (67)

1-(4-Bromobutoxy)-4-nitrobenzene (67) was synthesized form p-nitrophenol (8.35 g, 60.0 mmol), 1,4-dibromobutane (19.5 g, 90 mmol): yield, 11.1 g (67.7%) as syrup; ¹H NMR (CDCl₃) δ 2.00-2.09 (4H, m, 2×CH₂), 3.50 (2H, t, J=6.32 Hz, CH₂Br), 4.10 (2H, t, J=6.04 Hz, CH₂), 6.94 (2H, J=9.40 Hz, ArH), 8.20 (2H, J=9.40 Hz, ArH). Anal Calcd. for C₁₄H₂₂BrN₂O₅·½H₂O: C, 54.71; H, 7.21; N, 9.11. Found. C, 54.58; H, 7.50; N, 9.00.

By following the same procedure as that for the synthesis of 4-{4-[bis-(2-hydroxyethyl)amino]butoxy}-10H-acridin-9-one (62), compounds 68-71 were prepared.

### Example 9

### 2-{(2-Hydroxyethyl)-[4-(3-nitrophenoxy)butyl]amino}ethanol (68)

2-{(2-Hydroxyethyl)-[4-(3-nitrophenoxy)butyl]amino}ethanol (68) was prepared from 1-(4-bromobutoxy)-3-nitrobenzene (66) (5.0 g, 21.7 mmol) and diethanolamine (6.85 g, 65.1 mmol): yield 5.23 g (79.3%) as syrup; ¹H NMR(CHCl₃-d) δ, 1.67 (2H, m, CH₂), 1.83 (2H, m, CH₂), 2.62 (2H, t, J=7.20 Hz, NCH₂), 2.67 (4H, t, J=5.4 Hz, 2×NCH₂), 3.62 (4H, m, 2×CH₂OH), 4.04 (2H, t, J=5.28 Hz, OCH₂), 7.21 (1H, dq, J=2.69 and J=8.31 Hz, ArH), 7.41 (1H, t, J=8.22 Hz, ArH), 7.69 (1H, s, ArH), 7.78 (1H, dq, J=2.69 and J=8.31 Hz, ArH). Anal. Calcd. for C₁₄H₂₂N₂O₅·1.8H₂O: C, 50.60; H, 7.28; N, 8.43. Found. C, 50.65; H, 7.02; N, 8.18.

### Example 10

### 2-{(2-Hydroxyethyl)-[4-(4-nitrophenoxy)butyl]amino}ethanol (69)

2-{(2-Hydroxyethyl)-[4-(4-nitrophenoxy)butyl]amino}ethanol (69) was prepared from 4-1-(4-bromobutoxy)-4-nitrobenzene (67) (10.2 g, 37.2 mmol) and diethanolamine (10.6 g, 111 mmol): yield 6.4 g (6.2%) as syrup. ¹H NMR (CDCl₃) δ 1.68 (2H, m, CH₂), 1.85 (2H, m, CH₂), 2.38 (2H, brs, 2×OH), 2.63 (2H, t, J=7.36 Hz, NCH₂), 2.68 (4H, t, J=5.36 Hz, 2×NCH₂), 3.64 (4H, t, J=5.28 Hz, 2×CH₂OH), 4.07 (2H, t, J=6.28 Hz, OCH₂), 6.95 (2H, d, J=9.00 Hz, 2×ArH), 8.20 (2H, d, J=9.00 Hz, 2×ArH). Anal. Calcd. for C₁₄H₂₂BrN₂O₅·½H₂O: C, 54.71; H, 7.21; N, 9.11. Found. C, 54.58; H, 7.50; N, 9.00.

### Example 11

### 2-[(2-Hydroxyethyl)-(3-nitrobenzyl)amino]ethanol (70)

2-[(2-Hydroxyethyl)-(3-nitrobenzyl)amino]ethanol (70) was prepared from 3-nitrobenzyl chloride (17.16 g, 10 mmol) and diethanolamine (40.25 g, 30 mmol) in diglyme (20 mL): yield 13.93 g (58%); mp 71-72° C.; ¹H NMR (CDCl₃) δ 2.73 (4H, t, J=5.25 Hz, 2×NCH₂), 3.66 (4H, t, J=5.25 Hz, 2×CH₂OH), 3.82 (2H, s, CH₂), 7.51 (1H, t, J=7.74 Hz, ArH), 7.71 (1H, dt, J=1.36, J=7.74 Hz, ArH), 8.12 (1H, dt, J=1.13, J=7.74.14 Hz, ArH), 8.21 (1H, t, J=1.36 Hz, ArH). Anal. Calcd. for C₁₁H₁₆N₂O₄: C, 54.99; H, 6.71; N, 11.66. Found. C, 55.03; H, 6.72; N, 11.63.

### Example 12

### 2-[(2-Hydroxyethyl)-(4-nitrobenzyl)amino]ethanol (71)

2-[(2-Hydroxyethyl)-(4-nitrobenzyl)amino]ethanol (71) was prepared from 4-nitrobenzyl chloride (18.87 g, 11 mmol) and diethanolamine (44.28 g, 3.3 mmol); yield, 21.29 g, (80.5%), mp 74-75° C.; ¹H NMR (CDCl₃) δ 2.75 (4H, t, J=4.71 Hz, 2×NCH₂), 2.89 (2H, brs, 2×OH), 3.67 (4H, brs, 2×CH₂), 3.84 (2H, s, CH₂), 7.55 (2H, d, J=8.25 Hz, 2×ArH), 8.19 (2H, J=8.25 Hz, 2×ArH). Anal. Calcd. for C₁₁H₁₆N₂O₄: C, 54.99; H, 6.71; N, 11.66. Found. C, 55.14; H, 6.74; N, 11.64.

By following the same procedure as that for the synthesis of 4-[4-bis(2-chloroethyl)-aminobutoxy]acridin-9-one (60), compounds 72-75 were synthesized:

### Example 13

### Bis-(2-chloroethyl)-[4-(3-nitrophenoxy)butyl]amine (72)

Bis-(2-chloroethyl)-[4-(3-nitrophenoxy)butyl]amine (72) was prepared from 2-{(2-hydroxyethyl)-[4-(3-nitrophenoxy)butyl]amino}ethanol (67) (5 g, 16.76 mmol), methanesulfonyl chloride (5.75 g, 50.28 mmol) and triethylamine (6.78 g, 67.0 mmol): yield 3.4 g (60%) as HCl salt; mp 120-121° C.; ¹H NMR (CHCl₃-d) δ, 1.97 (2H, m, CH₂), 2.15 (2H, m, CH₂), 3.37 (2H, t, J=8.2 Hz, NCH₂), 3.57 (4H, s, 2×NCH₂), 4.10 (6H, m, OCH₂ and 2×CH₂Cl), 7.22 (1H, dq, J=8.31 Hz, ArH), 7.44 (1H, t, J=2.69 and J=8.24 Hz, ArH), 7.77 (1H, s, ArH), 7.84 (1H, dq, J=2.69 and J=8.31 Hz, ArH). Anal. Calcd. for C₁₄H₂₀N₂O₃Cl₂·0.5HCl·0.8H₂O: C, 45.55; H, 6.03; N, 7.58. Found. C, 45.55; H, 5.76; N, 7.50.

### Example 14

### Bis-(2-Chloroethyl)-[4-(4-nitrophenoxy)butyl]amine (73)

Bis-(2-Chloroethyl)-[4-(4-nitrophenoxy)butyl]amine (73) was prepared from 2-{(2-hydroxyethyl)-[4-(4-nitrophenoxy)butyl]amino}ethanol (69) (4.12 g, 13.8 mmol), methanesulfonyl chloride (4.70 g, 41.4 mmol) and triethylamine (5.59 g, 55.2 mmol); yield 3.96 g (85%) as syrup; mp 166-167° C. (HCl-salt); ¹H NMR (DMSO-d₆+D₂O) δ 1.81-1.82 (4H, m, 2×CH₂), 3.25 (2H, brs, NCH₂), 3.54 (4H, brs, 2×NCH₂), 4.03 (4H, t, J=6.40 Hz, 2×CH₂Cl), 4.16 (2H, t, J=6.40 Hz, OCH₂), 7.15 (2H, d, J=7.58 Hz, 2×ArH), 8.21 (2H, d, J=7.58 Hz, 2×ArH). Anal. Calcd. for C₁₄H₂₀Cl₂N₂O₃·HCl: C, 45.07; H, 5.67; N, 7.53. Found. C, 45.25; H, 5.75; N, 7.50.

### Example 15

### Bis-(2-Chloroethyl)-(3-nitrobenzyl)amine (74)

Bis-(2-Chloroethyl)-(3-nitrobenzyl)amine (74) was prepared from 2-[(2-hydroxyethyl)-(3-nitrobenzyl)amino]ethanol (70) (2.40 g, 10 mmol), methanesulfonyl chloride (2.86 g, 25 mmol) and triethylamine (3.05 g, 30 mmol): yield, 2.47 g (89%); mp 153-154° C.; ¹H NMR (CDCl₃) δ 2.96 (4H, t, J=6.73 Hz, 2×NCH₂), 3.54 (4H, t, J=6.73 Hz, 2×CH₂Cl), 3.87 (2H, s, CH₂), 7.51 (1H, t, J=7.85 Hz, ArH), 7.74 (1H, d, J=7.12 Hz, ArH), 8.13 (1H, s, ArH). Anal. Calcd. for C₁₁H₁₄Cl₂N₂O₂·HCl: C, 42.13; H, 4.82; N, 8.93. Found. C, 41.29; H, 4.82; N, 8.80.

### Example 16

### Bis-(2-Chloroethyl)-(4-nitrobenzyl)amine (75)

Bis-(2-Chloroethyl)-(4-nitrobenzyl)amine (75) was prepared from crude 2-[(2-hydroxyethyl)-(4-nitrobenzyl)amino]ethanol (71) (12.0 g, 50 mmol), methanesulfonyl chloride (17.18 g, 150 mmol) and triethylamine (20.23 g, 200 mmol); yield, 10.1 g (73%); mp 45-46° C.; ¹H NMR (CDCl₃) δ 2.95 (4H, t, J=6.73 Hz, 2×NCH₂), 3.53 (4H, J=6.73 Hz, CH₂Cl), 3.87 (2H, s, CH₂), 7.56 (2H, d, J=8.45, 2×ArH), 8.19 (2H, d, J=8.45 Hz, 2×ArH). Anal. Calcd. For C₁₁H₁₄Cl₂N₂O₂: C, 47.67; H, 5.09; N, 10.11. Found. C, 47.36; H, 5.10; N, 9.97.

### Example 17

### (3-(Acridin-9-ylamino)-5-{2-[bis(2-chloroethyl)amino]ethoxy}phenyl)methanol (37)

### 3-(Acridin-9-ylamino)-5-hydroxymethylphenol (76)

A solution of 9-chloroacridine (8.56 g, 40.0 mmol) in CHCl₃ (30 mL) was added dropwise to a solution of 3-amino-5-hydroxymethylphenol (7.03 g, 40 mmol) and 4-methylmorpholine (4.2 ml g, 40 mmol) in EtOH (150 mL) at −5° C. during 2.5 h. The reaction mixture was stirred for additional 1 h in an ice bath and then concentrated in vacuo to dryness and the residue was crystallized from ethanol to give 76; 4.73 g. Additional product, 4.97 g, was obtained from mother liquid after chromatography (SiO₂, 6×30 cm, solvent: CHCl₃: MeOH, 10:1 v/v); total 9.69 g (75.6%); mp 201-202° C.; ¹H NMR (DMSO-d₆) δ 4.43 (2H, s. CH₂), 5.23 (1H, brs, exchangeable, OH), 6.69 (1H, s, ArH), 6.80 (1H, s, ArH), 6.83 (1H, s, ArH), 7.45 (2H, m, 2×ArH), 7.99 (2H, m, 2×ArH), 8.10 (2H, m, 2×ArH), 8.29 (2H, m, 2×ArH), 9.88 (1H, brs, exchangeable, NH or OH), 11.43 (1H, br, exchangeable, NH or OH). Anal. Calcd. for C₂₀H₁₅N₂O₂·HCl·0.6H₂O: C, 65.84; H, 5.07; N, 7.68. Found. C, 65.86; H, 5.12; N, 7.51.

### (3-(Acridin-9-ylamino)-5-{2-[bis(2-chloroethyl)amino]ethoxy}phenyl)methanol (37)

A solution of (3-(acridin-9-ylamino)-5-hydroxymethylphenol (76) (2.68 g, 8.46 mmol) in 0.2 N KOH/MeOH (42.3 mL, 8.46 mmol) was stirred at room temperature of 10 min and resulted orange potassium salt was collected by filtration and dried. The salt was added into a mixture of tris(2-chloroethyl)amine hydrochloride (3.03 g, 12.6 mmol)ⱼ dry powdered K₂CO₃ (5.80 g, 42 mmol) and dry KF (0.487 mg, 8.46 mmol) in dry DMF (50 ml). The mixture was then gradually heated at 50° C. for 19 h and the filtered through a pad of celite, washed with DMF (10 mL). The combined filtrate and washing was evaporated in vacuo to dryness. The residue was dissolved in EtOAc (150 mL), washed with water (50 mL×3), dried over Na₂SO₄ and evaporated in vacuo to dryness. The residue was chromatographed on a silica gel column (4×17 cm) using CHCl₃ as the eluent. The product was eluted from CHCl₃/MeOH (10:1 v/v). The main fractions containing product were collected and evaporated in vacuo to dryness and the solid residue was recrystallized from acetone/hexane (5:1) to give 37, 2.04 g (50%); mp 153-154° C.; ¹H NMR (DMSO-d₆) δ 2.93 (6H, brs, 3×NCH₂), 3.60 (4H, brs, 2×CH₂Cl), 3.96 (2H, brs, OCH₂), 4.43 (2H, s, CH₂OH), 5.05 (1H, s, OH), 6.13 (1H, m, ArH), 6.28 (1H, m, ArH), 6.82 (1H, s, ArH), 7.00-7.12 (1H, m, ArH), 7.29 (2H, m, ArH), 7.46 (3H, m, 3×ArH), 7.72 (1H, m, ArH), 8.13 (1H, m, ArH), 8.16 (2H, m, ArH), 10.84 (1H, brs, exchangeable, NH). Anal. Calcd. for C₂₆H₂₇Cl₂N₃O₂: C, 64.24; H, 5.62; N, 8.66. Found. C, 64.20; H, 5.88; N, 8.28.

### Example 18

### Acridin-9-yl-(3-{2-[bis(2-chloroethyl)amino]ethoxy}phenyl)amine (38)

A mixture of bis-(2-chloroethyl)-[2-(3-nitrophenoxy)ethyl]amine (64) (307 mg, 1.0 mmol) and SnCl₂·H₂O (675 mg, 3 mmol) in conc. HCl (4 mL) was stirred at 60° C. for 30 mm. The clear solution was poured into ice (25 g) and neutralized slowly with NH₄OH (25%). The mixture was extracted with CHCl₃ (4×50 mL), dried over Na₂SO₄ and evaporated in vacuo to dryness to give crude 3-bis-(2-chloroethyl)aminoethoxyaniline, which was dissolved in CHCl₃ (20 mL) and added to a solution of 9-chloroacridine (106 mg, 0.5 mmol) and 2 drops of conc. HC1 in CHCl₃ (20 mL) in an ice bath. After being stirred at room temperature for 6 h, the mixture was evaporated in vacuo to dryness and the residue was chromatographed on silica gel column (1×20 cm) using CHCl₃: methane (10:1 v/v) as the eluant. The main fractions containing the desired product were collected and evaporated in vacuo to dryness. The residue was treated with excess 2.5M HCl/EtOAc and evaporated under reduced pressure to dryness and the solid residue was recrystallized from acetone/ethyl acetate to give 38, 164 mg (36%) as HCl salt; mp 123-124° C.; ¹H NMR (DMSO-d₆) δ 2.97 (6H, t, J=7.09 Hz, 3×NCH₂), 3.49 (4H, t, J=7.09 Hz, 2×CH₂Cl), 3.94 (2H, t, J=5.38 Hz, OCH₂), 6.51 (1H, s, ArH), 6.56 (1H, m, ArH), 7.17 (1H, m, ArH), 7.22 (2H, brs, ArH), 7.60 (2H, m, ArH), 7.94 (2H, m, ArH), 8.04 (2H, m, ArH), 11.21 (1H, brs, NH). Anal. Calcd. for C₂₅H₂₅Cl₂N₃O·1·HCl·5H₂O: C, 51.68; H, 6.25; N, 7.23. Found. C, 51.70; H, 6.30; N, 7.26.

By following the same procedure as that for the synthesis of compound 38 (Method 2), compounds 39-41 (Type 1) were prepared:

### Example 19

### Acridin-9-yl-(3-{4-[bis(2-chloroethyl)amino]butoxy}phenyl)amine (39)

Acridin-9-yl-(3-{4-[bis(2-chloroethyl)amino]butoxy}phenyl)amine (39) was prepared from bis-(2-chloroethyl)-[4-(3-nitrophenoxy)butyl]amine (72) (335 mg, 1.0 mmol) and 9-chloroacridine (106.8 mg, 0.5 mmol): yield 149 mg (30%); mp 126-127° C.; ¹H NMR (DMSO-d₆) δ 1.81 (2H, m, NCH₂), 1.91 (2H, m, OCH₂), 3.31 (2H, m, NCH₂), 3.56 (4H, m, 2×NCH₂), 4.08 (6H, m, 2×CH₂Cl and OCH₂), 7.10 (2H, m, 2×ArH), 7.42 (3H, m, 3×ArH), 7.43 (1H, s, ArH), 7.99 (2H, m, 2×ArH), 8.08 (2H, m, 2×ArH), 8.25 (2H, m, 2×ArH), 8.04 (2H, m, ArH), 11.45 (1H, brs, exchangeable, NH).

Calcd. for C₂₇H₂₉Cl₂N₃O·2·HCl·5H₂O; C, 50.24; H, 6.40; N, 6.51. Found. C, 50.61; H, 6.53; N, 6.27.

### Example 20

### Acridin-9-yl-(4-{2-[bis(2-chloroethyl)amino]ethoxy}phenyl)amine (40)

Acridin-9-yl-(4-{2-[bis(2-chloroethyl)amino]ethoxy}phenyl)amine (40) was prepared from bis-(2-chloroethyl)-[2-(4-nitrophenoxy)ethyl]amine (65) (921 mg, 3.0 mmol) and 9-chloroacridine (544 mg, 2.55 mmol): yield 390 mg (29%); mp 225-226° C.; ¹H NMR (DMSO-d₆+D₂O) δ 3.62 (6H, s, 3×NCH₂), 4.08 (4H, s, 2×CH₂Cl), 4.47 (2H, s, OCH₂), 7.12 (2H, m, 2×ArH), 7.40-7.46 (4H, m, 4×ArH), 7.91 (2H, m, 2×ArH), 8.16 (2H, m, 2×ArH), 8.29 (2H, m, 2×ArH), 11.70 (1H, brs, NH). Anal. Calcd. for C₂₅H₂₅Cl₂N₃O·2HCl·0.5H₂O: C, 55.98; H, 5.26; N, 7.84. Found. C, 56.06; H, 5.33; N, 7.84.

### Example 21

### Acridin-9-yl-(4-{4-[bis(2-chloroethyl)amino]buoxy}phenyl)amine (41)

Acridin-9-yl-(4-{4-[bis(2-chloroethyl)amino]buoxy}phenyl)amine (41) was prepared from of 2-{(2-chloroethyl)-[4-(4-nitrophenoxy)butyl]amino}ethanol (72) (892 mg, 2.4 mmol) and 9-chloroacridine (213 mg, 1.0 mmol): yield 110 mg (22%); mp 109-110° C.; ¹H NMR (DMSO-d₆) δ 1.81 (4H, m, 2×CH₂), 3.29 (2H, m, NCH₂), 4.08 (6H, m, 2×CH₂Cl₂ and OCH₂), 7.10 (2H, m, 2×ArH), 7.41 (1H, m, 4×ArH), 7.89 (2H, m, 2×ArH), 8.08 (2H, m, 2×ArH), 8.27 (2H, m, 2×ArH), 11.36 (1H, brs, NH). Anal. Calcd. for C₂₇H₂₉Cl₂N₃O·3HCl·0.5H₂O: C, 53.97; H, 5.53; N, 6.99. Found. C, 53.34; H, 5.43; N, 7.03.

### Example 22

### 9-(3-{2-[Bis(2-chloroethyl)amino]ethoxy}phenylamino)-5-methylacridin-4-carboxylic acid (2-dimethylaminoethyl)amide (42)

Tin(II) chloride dihydrate (675 mg, 3.0 mmol) was added portionwise to a suspension of bis(2-chloroethyl)-[2-(3-nitrophenoxy)ethyl]amine (64) (307 mg, 1.0 mmol) in conc. HCl (5 ml). The reaction mixture was heated at 60° C. for 20 min. The mixture was poured into ice (30 g), neutralized with NH₄OH (25%) and then extracted with CHCl₃ (3×25 mL). The organic extracts were combined, washed with water (4×15 mL), dried over Na₂SO₄, and concentrated in vacuo to give crude bis(2-chloroethyl)-[2-(3-aminophenoxy)ethyl]amine (318 mg), which was dissolved in CHCl₃ (20 mL) and then added into a solution of 9-chloro-5-methylacridan-4-dimethylaminoethylcarboxamide (274 mg, 0.8 mmol) in CHCl₃ (20 mL) containing one drops of conc. HCl at −5° C. The reaction mixture was continuously stirred at room temperature overnight and then evaporated in vacuo to dryness. The residue was chromatographed on a silica gel column (1×20 cm) using CHCl₃/MeOH (10:1 v/v) as the eluant. The fractions containing the product were collected and concentrated in vacuo and the residue dissolved in 4.2 N HCl/ethyl acetate (3 mL) and evaporated in vacuo to dryness. The solid residue was recrystallized from ethyl acetate/acetone to give 38.24 mg of 42 (41%). mp 129-130° C.; ¹H NMR (DMSO-d₆) δ 2.73 (3H, s, Me), 2.87 and 2.88 (each 3H, s, NMe₂), 3.16 (2H, brs, NCH₂), 3.66 (2H, brs, NCH₂), 3.82 (4H, m, 2×CH₂Cl), 4.24 (2H, m, OCH₂), 7.10-7.13 (1H, m, ArH), 7.17 (1H, m, ArH), 7.39-7.43 (3H, m, 3ArH), 7.53-7.55 (1H, m, ArH), 7.91 (1H, m, ArH), 8.14 (1H, m, ArH), 8.52 (1H, m, ArH), 8.82 (1H, d, ArH), 9.92 (1H, brs, NH), 10.51 (1H, brs, NH). Anal. Calcd. for C₃₁H₃₇N₅O₂Cl₂·4HCl·3H₂O: C, 47.60; H, 6.06; N, 8.95. Found. C, 47.60; H, 6.45; N, 8.91.

By following the same procedure as that for the synthesis of 38, compounds 43-45 were prepared.

### Example 23

### 9-(3-{4-[Bis(2-chloroethyl)amino]butoxy}phenylamino)-5-methylacridin-4-carboxylic acid (2-dimethylaminoethyl)amide (43)

9-(3-{4-[Bis(2-chloroethyl)amino]butoxy}phenylamino)-5-methylacridin-4-carboxylic acid (2-dimethylaminoethyl)amide (43) was prepared from bis-(2-chloroethyl)-[4-(3-nitrophenoxy)butyl]amine (72) (335 mg, 1.0 mmol) and 9-chloro-5-methylacridan-4-dimethylaminoethylcarboxamide (171 mg, 0.5 mmol): yield 156 mg (26.8%); mp 131-132° C.; ¹HNMR (DMSO-d₆) δ 1.70 (4H, m, 2×CH₂), 2.86 (9H, s, Me and 2×NCH₃), 3.50 (2H, m, NCH₂), 3.58 (6H, m, 3×NCH₂), 3.79 (2H, m, CH₂), 3.86 (2H, m, OCH₂), 3.95 (4H, m, 2×CH₂Cl), 6.43 (1H, m, ArH), 6.52 (1H, m, ArH), 6.79 (1H, brs, exchangeable, NH), 7.10 (1H, m, ArH), 7.32 (1H, m, ArH), 7.42 (1H, m, ArH), 7.58 (1H, m, ArH), 7.77 (1H, m, ArH), 8.09 (1H, m, ArH), 8.42 (1H, m, ArH), 8. 69 (1H, m, ArH). Anal. Calcd. for C₃₃H₄₁Cl₂N₅O₂·3HCl·7H₂O; C, 46.84; H, 6.91; N, 8.28. Found. C, 46.91; H, 6.75; N, 8.22.

### Example 24

### 9-(4-{2-[Bis(2-chloroethyl)amino]ethoxy}phenylamino)-5-methylacridin-4-carboxylic acid (2-dimethylaminoethyl)amide (44)

9-(4-{2-[Bis(2-chloroethyl)amino]ethoxy}phenylamino)-5-methylacridin-4-carboxylic acid (2-dimethylaminoethyl)amide (44) was prepared from bis-(2-chloroethyl)-[2-(4-nitrophenoxy)ethyl]amine (65) (377 mg, 1.1 mmol) and 9-chloro-5-methylacridan-4-dimethylaminoethylcarboxamide (274 mg, 0.8 mmol): yield 307 mg (67%); mp 185-186° C.; ¹H NMR (DMSO-d₆) δ 2.72 (3H, s, Me), 2.86 (3H, brs, NMe), 2.87 (3H, s, NMe), 3.05-3.16 (6H, m, 3×NCH₂), 3.40 (2H, m, NCH₂), 3.69-3.81 (6H, m, 2×CH₂Cl and NCH₂), 4.22 (2H, brs, OCH₂), 7.11 (2H, d, 2×ArH), 7.36-7.42 (3H, m, 3×ArH), 7.54 (1H, m, ArH), 7.91 (1H, m, ArH), 8.17 (1H, m, ArH), 8.54 (1H, m, ArH), 8.84 (1H, m, ArH), 9.97 (1H, brs, NH), 10.74 (1H, brs, NH). Anal. Calcd. for C₃₁H₃₇Cl₂N₅O₂·2.5HCl·4H₂O: C, 49.86; H, 6.41; N, 9.38. Found. C, 50.21; H, 6.67; N, 9.65.

### Example 25

### 9-(4-{4-[Bis(2-chloroethyl)amino]butoxy}phenylamino)-5-methylacridin-4-carboxylic acid (2-dimethylaminoethyl)amide (45)

9-(4-{4-[Bis(2-chloroethyl)amino]butoxy}phenylamino)-5-methylacridin-4-carboxylic acid (2-dimethylaminoethyl)amide (45) was prepared from bis-(2-chloroethyl)-[4-(4-nitrophenoxy)butyl]amine (72) (892 mg, 2.4 mmol) and 9-chloro-5-methylacridan-4-dimethylaminoethylcarboxamide (410 mg, 1.2 mmol): yield 436 mg (71%); mp 166-167° C.; ¹H NMR (DMSO-d₆) δ 1.81 (2H, m, CH₂), 1.90 (2H, m, CH₂), 2.72 (3H, s, Me), 2.86 (3H, s, NMe), 2.87 (3H, s, NMe), 3.29 (2H, br, NCH₂), 3.40 (2H, m, NCH₂), 3.58 (4H, m, 2×NCH₂), 3.82 (2H, m, NCH₂), 4.07 (6H, m, 2×CH₂Cl+OCH₂), 7.10 (2H, m, 2×ArH), 7.37-7.42 (3H, m, 3×ArH), 7.55 (1H, m, ArH), 7.91 (1H, m, ArH), 8.16 (1H, m, ArH), 8.53 (1H, m, ArH), 8.83 (1H, m, ArH), 9.90 (1H, brs, NH), 10.68 (1H, brs, NH). Anal. Calcd. for: C₃₃H₄₁Cl₂N₅O₂·1.5HCl·4H₂O: C, 44.87; H, 6.21; N, 7.92. Found. C, 44.95; H, 6.24; N, 7.84.

### Example 26

### Acridin-9-yl-(3-{[bis-(2-chloroethyl)amino]methyl}phenyl)amine (46)

Acridin-9-yl-(3-{[bis(2-chloroethyl)amino]methyl}phenyl)amine (46) was prepared from bis-(2-chloroethyl)-(3-nitrobenzyl)amine (74) (1.11, 4.0 mmol) and 9-chloroacridine (0.76 g, 3.55 mmol): yield 1.44 g (85%); mp 207-208° C.; ¹H NMR (DMSO-d₆) ¹H NMR (DMSO-d₆) δ 3.30 (4H, t, J=6.41 Hz, 2×NCH₂), 3.89 (4H, t, J=6.41 Hz, 2×CH₂Cl), 4.27 (2H, s, CH₂), 7.48 (2H, m, 2×ArH), 7.52 (1H, m, ArH), 7.61 (3H, m, 3×ArH), 8.06 (4H, m, 4×ArH), 8.25 (2H, m, 2×ArH), 11.78 (1H, brs, NH). Anal. Calcd. for C₂₄H₂₃Cl₂N₃·2HCl·0.5H₂O: C, 56.93; H, 5.18; N, 8.30. Found. C, 55.63; H, 5.20; N, 8.29.

### Example 27

### Acridin-9-yl-(4-{[bis(2-chloroethyl)amino]methyl}phenyl)amine (47)

Acridin-9-yl-(4-{[bis(2-chloro ethyl)amino]methyl}phenyl)amine (47) was prepared from bis(2-chloroethyl)-(4-nitrobenzyl)amine (75) (1.11 g, 4.0 mmol) and 9-chloroacridine (0.629 g, 3.0 mmol): yield 0.589 (35%); mp 220-223° C.; ¹H NMR (DMSO-d₆) δ 3.43 (4H, m, 2×NCH₂); 4.01 (4H, brs, 2×CH₂Cl); 4.40 (2H, brs, CH₂); 7.44 (2H, m, ArH); 7.51 (2H, m, ArH); 7.72 (2H, m, ArH); 8.03 (2H, m, ArH); 8.19 (2H, m, ArH); 8.28 (2H, m, ArH); 11.74 (1H, brs, NH). Anal. Calcd. for C₂₄H₂₃Cl₂N₃·2HCl·0.5H₂O: C, 56.93; H, 5.18; N, 8.30. Found. C, 56.86; H, 5.31; N, 8.16.

### Example 28

### 9-(3-{[Bis(2-chloroethyl)amino]methyl}phenylamino)-5-methylacridine-4-carboxylic acid (2-dimethylaminoethyl)amide (48)

9-(3-{[Bis(2-chloroethyl)amino]methyl}phenylamino)-5-methylacridine-4-carboxylic acid (2-dimethylaminoethyl)amide (48) was prepared from bis(2-chloroethyl)-(3-nitrobenzyl)amine (74) (555 mg, 2.0 mmol) and 9-chloro-5-methylacridan-4-dimethylaminoethylcarboxamide (652 mg, 1.99 mmol): yield 1.02 g (92.13%); mp 198-199° C.; ¹H NMR (DMSO-d₆) δ 2.76 (3H, s, Me), 2.92 (6H, s, 3×NMe₂), 3.25 (4H, s, 2×NCH₂), 3.44 (2H, brs, CH₂), 3.85 (6H, m, 2×CH₂Cl; and CH₂), 4.22 (2H, s, CH₂), 7.43 (1H, m, ArH), 7.49 (1H, m, ArH), 7.57 (4H, 4×ArH), 7.97 (1H, m, ArH), 8.11 (1H, m, ArH), 8.53 (1H, m, ArH), 8.77 (1H, m, ArH), 10.05 (1H, s, NH), 10.82 (1H, s, NH). Anal. Calcd. for C₃₀H₃₅Cl₂N₅O·3HCl·2H₂O: C, 51.62; H, 6.07; N, 10.03. Found. C, 51.50; H, 6.20; N, 10.19.

### Example 29

### 9-(4-{[Bis(2-chloroethyl)amino]methyl}phenylamino)-5-methylacridin-4-carboxylic acid (2-dimethylaminoethyl)amide (49)

9-(4-{[Bis(2-chloroethyl)amino]methyl}phenylamino)-5-methylacridan-4-carboxylic acid (2-dimethylaminoethyl)amide (49) was prepared from bis(2-chloroethyl)-(4-nitrobenzyl)amine (75) (555 mg, 2.0 mmol) and 9-chloro-5-methylacridan-4-dimethylaminoethylcarboxamide (652 mg, 1.99 mmol): yield 0.84 g (76.3%); mp 204-205° C.; ¹H NMR (DMSO-d₆) δ 2.73 (3H, s, Me), 2.87 and 2.88 (each 3H, s, NMe₂), 3.43 (6H, m, 3×NCH₂), 3.84 (2H, CH₂), 3.96 (4H, 2×CH₂Cl), 4.46 (2H, s, CH₂), 7.39 (1H, ArH), 7.53 (3H, m, ArH), 7.76 (2H, brs, ArH), 7.93 (1H, m, ArH), 8.20 (1H, m, ArH), 8.57 (1H, m, ArH), 8.92 (1H, m, ArH), 10.08 (1H, brs, NH), 10.86 (1H, brs, NH). Anal. Calcd. for C₃₀H₃₅Cl₂N₅O·3HCl·2H₂O: C, 51.62; H, 6.07; N, 10.03. Found. C, 51.39; H, 6.26; N, 9.97.

### Example 30

### [3-Amino-5-(4-{2-[bis(2-chloroethyl)amino]ethoxy}acridin-9-ylamino)phenyl]methanol (50)

A mixture of 4-{2-[bis(2-chloroethyl)amino]ethoxy}-10H-acridine-9-one (60) (1.52 g, 4.0 mmol), SOCl₂ (5 mL) containing 2 drops of DMF was heated to 80° C. for 40 min. The mixture was evaporated under reduced pressure to dryness. The residue was co-evaporated with CHCl₃ (20 mL×3) to yield crude 4-{2-[bis(2-chloroethyl)-amino]ethoxy}-9-chloroacridine, which was dissolved in CHCl₃ (25 mL) and filtered to remove insoluble by-products. The filtrate was then added dropwise to a solution of 3,5-diaminobenzyl alcohol dihydrochloride (912 mg, 4.2 mmol) containing conc. HCl (0.5 mL) in EtOH (600 ml) in an ice-bath during 1 h. The reaction mixture was then stirred at 0° C. for additional 3 h and then evaporated in vacuo to dryness. The residue was chromatographed on a silica gel column (6×25 cm) using CHCl₃/MeOH (100:30 v/v) as the eluant. The fractions containing the product were combined and evaporated under reduced pressure and the residue was treated with excess 2.5M HCl/EtO and evaporated under reduced pressure. The residue was crystallized from ethanol/aceton to give 50, 1.68 g (67%); mp 105-106° C.; ¹H NMR (DMSO-d₆) δ 3.79 (4H, s, 2×NCH₂), 3.99 (2H, s, NCH₂), 4.20 (4H, s, CH₂Cl×2), 4.46 (2H, s, ArCH₂), 4.74 (2H, s, OCH₂), 7.18 (1H, s, ArH), 7.26 (2H, s, ArH), 7.42-7.51 (2H, m, ArH), 7.60 (1H, m, ArH), 7.95 (1H, m, ArH), 8.03 (1H, m, ArH), 8.35 (1H, m, ArH), 8.99 (1H, m, ArH), 11.96 (1H, brs, NH). Anal. Calcd. for C₂₆H₂₈Cl₂N₄O₂·4HCl·3H₂O: C, 44.65; H, 5.47; N, 8.01. Found. C, 44.63; H, 5.28; N, 7.87.

### Example 31

### [3-(4-{2-[Bis-(2-[bis-(2-chloroethyl)amino]ethoxy)acridin-9-ylamino]-5-hydroxymethylphenyl}carboxylic acid ethyl ester (51)

Ethyl chloroformate (64.9 mg, 0.6 mmol) was added dropwise to a mixture of [3-amino-5-(4-{2-[bis(2-chloroethyl)amino]ethoxy}acridin-9-ylamino)phenyl]methanol (50) (250 mg, 0.5 mmol) and pyridine (47.5 mg, 0.6 mmol) in dry DMF (15 mL) in an ice-bath. After being stirred for 40 min, the reaction mixture was evaporated in vacuo to dryness and the residue was chromatographed on a silica gel column (2×20 cm) using CHCl₃/MeOH (10:1 v/v) as the eluant. The desired product 51, 249 mg (72%), was obtained after recrystallization from EtOAc/EtOH, mp 151-152° C.; ¹H NMR (DMSO-d₆+D₂O) δ 1.27 (3H, t, J=7.24 Hz, Me), 3.08 (4H, t, J=6.60 Hz, 2×NCH₂) 3.17 (2H, s, NCH₂), 3.62 (4H, t, J=6.64 Hz, 2×CH₂Cl), 4.11-4.21 (4H, m, CH₂+OCH₂), 4.61 (2H, s, CH₂), 6.56 (1H, s, NH), 6.59 (1H, s, ArH), 6.70 (1H, s, ArH), 6.82-7.10 (3H, m, ArH), 7.19 (1H, s, ArH), 7.45 (2H, m, NH), 8.01 (1H, brs, ArH). Anal. Calcd. for C₂₉H₃₂Cl₂N₄O₄: C, 60.94; H, 5.64; N, 9.80. Found. C, 60.66; H, 5.70; N, 9.58.

By following the same procedure as that for the synthesis of 50, compounds 52 and 53 were synthesized:

### Example 32

### [3-Amino-5-(4-{4-[bis-(2-chloroethyl)amino]butoxy}acridin-9-ylamino)phenyl]-methanol (52)

[3-Amino-5-(4-{4-[bis-(2-chloroethyl)amino]butoxy}acridin-9-ylamino)phenyl]methanol (52) was prepared from 4-{4-[bis-(2-chloroethyl)amino]butoxy}-10H-acridin-9-one (63) (815 mg, 2.0 mmol), 3,5-diaminobenzyl alcohol dihydrochloride (317 mg, 1.5 mmol) and 4-N-methylmorpholine (0.31 mL, 2.8 mmol): yield 290 mg (27%); mp 247-248° C.; ¹H NMR (DMSO-d₆) δ 2.04 (4H, m, 2×CH₂), 3.35 (2H; brs; NCH₂); 3.57 (4H; t; J=7.00 Hz, 2×NCH₂), 4.12 (4H; t; J=7.08 Hz, 2×CH₂Cl), 4.41 (2H, brs, OCH₂); 4.46 (3H, brs, CH₂OH and OH), 7.05 (1H, s, ArH), 7.08 (1H, s, ArH); 7.11 (1H, s, ArH), 7.41 (1H, m, ArH), 7.49 (1H, m, ArH), 7.57 (1H, m, ArH), 7.86 (1H, m, ArH), 8.01 (1H, m, ArH), 8.32 (1H, m, ArH), 8.77 (1H, m, ArH), 11.66 (1H, brs, NH). Anal. Calcd. for C₂₈H₃₂Cl₂N₄O₂·3HCl·1.5H₂O: C, 50.65; H, 5.77; N, 8.44; Found. C, 50.95; H, 5.72; N, 8.40.

### Example 33

### 3-(4-{2-[Bis-(2-chloroethyl)amino]ethoxy}acridin-9-ylamino)-5-hydroxymethylphenol (53)

3-(4-{2-[Bis(2-chloroethyl)amino]ethoxy}acridin-9-ylamino)-5-hydroxymethylphenol (53) was prepared from 4-{4-[bis(2-chloroethyl)amino]butoxy}-10H-acridin-9-one (64) (1.90 g, 5.0 mmol) and 3,5-diaminobenzyl alcohol (685 mg, 5.0 mmol); yield 1.73 (73%); mp 237-238° C.; ¹H NMR (DMSO-d₆+D₂O) δ 3.80-3.36 (6H, m, NCH₂×3), 4.09 (4H, s, 2×CH₂Cl), 4.57 (2H, s, ArCH₂), 4.67 (2H, s, CH₂), 6.71 (1H, s, ArH), 6.88 (1H, s, ArH), 6.90 (1H, s, ArH), 7.42-7.40 (3H, m, ArH), 7.58 (1H, m, ArH), 7.85 (1H, m, ArH), 7.97 (1H, m, ArH), 8.20 (1H, m, ArH), 8.50 (1H, brs, NH). Anal. Calcd. for C₂₆H₂₇Cl₂N₃O₃·4HCl·3H₂O: C, 44.65; H, 5.47; N, 8.01. Found. C, 44.63; H, 5.18; N, 7.77.

By following the same procedure as that for the synthesis of 38 and 50, compounds 54-58 were prepared:

### Example 34

### (4-{2-[Bis-(2-chloroethyl)amino]ethoxy}acridin-9-yl)-(3-{2-[bis(2-chloroethyl)-amino]ethoxy}phenyl)amine (54)

(4-{2-[Bis(2-chloroethyl)amino]ethoxy}acridin-9-yl)-(3-{2-[bis(2-chloroethyl)-amino]ethoxy}phenyl)amine (54) was prepared from bis(2-chloroethyl)-[2-(3-nitrophenoxy)ethyl]amine (64) (307 mg, 1.0 mmol) and 4-{2-[bis(2-chloroethyl)-amino]ethoxy}-10H-acridine-9-one (60) (205 mg, 0.6 mmol): yield 252 mg (39.5%);

mp: 108-109° C.; ¹H NMR (DMSO-d₆) δ 3.77 (6H, m, 3×NCH₂), 3.95 (2H, m, CH₂N), 4.06 (4H, t, J=7.09 Hz, 2×CH₂Cl₂), 418 (4H, t, J=7.09 Hz, 2×CH₂Cl₂), 4.46 (2H, s, OCH₂), 4.71 (2H, s, OCH₂), 7.13 (2H, m, ArH), 7.43 (4H, m, 3×ArH), 7.57 (1H, m, ArH), 7.89 (1H, m, ArH), 7.98 (1H, m, ArH), 8.26 (1H, m, ArH), 8.33 (1H, m, ArH), 8.89 (1H, m, ArH), 11.76 (1H, brs, exchangeable, NH). Anal. Calcd. for C₃₁H₃₆Cl₄N₄O₂·3HCl·4·H₂O: C, 45.41; H, 5.78; N, 6.83. Found. C, 45.32; H, 5.72; N, 6.50.

### Example 35

### (3-{4-[Bis(2-chloroethyl)amino]butoxy}phenyl-(4-{2-[bis(2-chloroethyl)amino]-ethoxy}acridin-9-yl)amine (55)

(3-{4-[Bis(2-chloroethyl)amino]butoxy}phenyl-(4-{2-[bis(2-chloroethyl)amino]-ethoxy}acridin-9-yl)amine (55) was prepared from bis(2-chloroethyl)-[4-(3-nitrophenoxy)butyl]amine (72) (153 mg, 0.5 mmol) and 4-{2-[bis(2-chloroethyl)-amino]ethoxy}-10H-acridine-9-one (60) (190 mg, 0.5 mmol): yield 242 mg (40%);

mp 98-99° C.; ¹H NMR (DMSO-d₆) δ 1.75 (2H, m, CH₂), 1.85 (2H, m, CH₂), 3.56 (4H, m, 2×NCH₂), 3.81 (4H, m, 2×NCH₂), 4.09 (6H, t, J=6.85 Hz, 2×CH₂Cl₂+OCH₂), 4.22 (4H, t, J=6.85 Hz, 2×CH₂Cl₂), 4.74 (2H, s, OCH₂), 6.99 (2H, m, ArH), 7.12 (1H, m, ArH), 7.37-7.48 (3H, m, 3×ArH), 7.58 (1H, m, ArH), 7.99 (2H, m, ArH), 8.37 (1H, m, ArH), 12.00 (1H, brs, NH).

### Example 36

### (4-[2-[Bis(2-chloroethyl)amino]ethoxy]acridin-9-yl)-(4-{2-[bis(2-chloroethyl)amino]-ethoxy}phenyl)amine (56)

(4-[2-[Bis(2-chloroethyl)amino]ethoxy]acridin-9-yl)-(4-{2-[bis(2-chloroethyl)amino]-ethoxy}phenyl)amine (56) was prepared from bis(2-chloroethyl)-[2-(4-nitrophenoxy)ethyl]amine (65) (617 mg, 1.8 mmol) and 4-{2-[bis(2-chloroethyl)amino]ethoxy}-10H-acridine-9-one (60) (597 mg, 1.5 mmol): yield 595 mg (62%); mp 105-106° C.; ¹H NMR (DMSO-d₆) δ 3.73 (6H, brs, 3×NCH₂), 3.90 (2H, brs, NCH₂, 4.01 (4H, m, 2×CH₂Cl), 4.15 (4H, m, 2×CH₂Cl), 4.43 (2H, brs, OCH₂), 4.69 (2H, brs, OCH₂), 7.14 (2H, d, J=8.48 Hz, ArH), 7.38-7.45 (4H, m, ArH), 7.58 (1H, m, ArH), 7.86 (1H, m, ArH), 7.99 (1H, m, ArH), 8.22 (1H, m, ArH), 8.84 (1H, m, ArH). Anal. Calcd. for C₃₁H₃₆Cl₄N₄O₂·6HCl·3H₂O: C, 40.85; H, 5.31; N, 6.14. Found. C, 40.87; H, 5.09; N, 6.03.

### Example 37

### (4-{4-[Bis(2-chloroethyl)amino]butoxy}acridin-9-yl)-(4-{2-[bis(2-chloroethyl)amino]-ethoxy}phenylamine (57)

(4-{4-[Bis(2-chloroethyl)amino]butoxy}acridin-9-yl)-(4-{2-[bis(2-chloroethyl)amino]ethoxy}phenylamine (57) was prepared from bis-(2-chloroethyl)-[2-(4-nitrophenoxy)ethyl]amine (65) (412 mg, 1.2 mmol) and 4-{4-[bis-(2-chloroethyl)-amino]butoxy}-10H-acridin-9-one (63) (407 mg, 1.0 mmol): yield 375 mg (63%); mp 101-102° C.; ¹H NMR (DMSO-d₆) δ 1.64-1.72 (2H, m, CH₂), 1.94-2.01 (2H, m, CH₂), 2.67 (2H, t, J=7.02 Hz, NCH₂), 2.90 (4H, t, J=8.91 Hz, 2×NCH₂), 3.02 (6H, m, 3×NCH₂), 3.50-3.65 (8H, m, 4×CH₂Cl), 4.03 (2H, t, J=5.6 Hz, OCH₂), 4.16 (2H, t, J=6.38 Hz, OCH₂), 6.77-6.87 (7H, m, 7×ArH), 7.01 (1H, m, ArH), 7.30 (1H, m, ArH), 7.40-7.44 (2H, m, ArH), 7.92 (1H, brs, NH). Anal. Calcd. for: C₃₃H₄₀Cl₄N₄O₂·8HCl·6H₂O: C, 39.81; H, 6.75; N, 5.63. Found. C, 39.63; H, 6.54; N, 5.66.

### Example 38

### (3-{4-[Bis-(2-chloroethyl)amino]butoxy}phenyl-(4-{2-[bis(2-chloroethyl)amino]-ethoxy}acridin-9-yl)amine (58)

(3-{4-[Bis(2-chloroethyl)amino]butoxy}phenyl-(4-{2-[bis(2-chloroethyl)amino]-ethoxy}acridin-9-yl)amine (58) was prepared from bis(2-chloroethyl)-[4-(4-nitrophenoxy)butyl]amine (73) (588 mg, 1.5 mmol) and 4-{2-[bis-(2-chloroethyl)-amino]ethoxy}-10H-acridine-9-one (60) (397.7 mg, 0.8 mmol): yield 115 mg (24%); mp 95-96° C.; ¹H NMR (DMSO-d₆) δ 1.87 (2H, brs, CH₂), 1.97 (2H, brs, CH₂), 3.36 (2H, brs, NCH₂), 3.63 (4H, brs, 2×NCH₂), 3.84 (4H, brs, 2×NCH₂), 4.01 (2H, brs, NCH₂), 4.16 (6H, brs, 2×CH₂Cl+OCH₂), 4.25 (4H, brs, 2×CH₂Cl), 4.77 (2H, brs, OCH₂), 7.12 (2H, m, ArH), 7.45-7.47 (5H, m, 5×ArH), 7.61 (1H, m, ArH), 8.02 (2H, m, ArH), 8.40 (1H, m, ArH), 8.99 (1H, m, NH). Anal. Calcd. for: C₃₃H₄₀Cl₄N₄O₂·7HCl·4H₂O: C, 42.9; H, 6.00; N, 6.07. Found. C, 43.63; H, 6.10; N, 6.02.

### Example 39

### {3-[5-(2-Dimethylaminoethylcarbamoyl)-1-methyl-1H-pyrrol-3-ylcarbamoyl]propyl}carbamic acid tert-butyl ester

A mixture of known 4-tert-butoxycarbonylaminobutyric acid 2,5-dioxopyrrolidin-1-yl ester (900 mg, 3.0 mmol), 4-amino-1-methyl-1H-pyrrole-2-carboxylic acid (2-dimethylamino)amide [freshly prepared from 4-nitro-1-methyl-1H-pyrrole-2-carboxylic acid (2-dimethyl-amino)amide, 718 mg, 3.0 mmol, 10% Pd/C/H₂, 336 mg in DMF (40 mL), 30 psi, 30 min], and triethylamine (304 mg. 3.0 mmol) was stirred at room temperature overnight. The reaction mixture was evaporated in vacuo to dryness and the residue was chromatographed on a silica gel column (4×25 cm) using CHCl₃/MeOH (5:1 v/v) as the eluent. The product was obtained as syrup, 1.13 g (95%); ¹H NMR (DMSO-d₆) 61.39 (9H, s, 3×Me), 1.63 (2H, m, CH₂), 2.42 (2H, t, J=6.74 Hz, CH₂), 2.92 (2H, m, CH₂), 3.02 (2H, m, CH₂), 3.26 (2H, m, CH₂), 3.77 (3H, s, Me), 6.77 (1H, s, ArH), 7.13 (1H, s, ArH). Anal. Calcd. for: C₁₉H₃₃N₅O₄: C, 57.70; H, 8.41; N, 17.71. Found C, 57.81; H, 8.36; N, 17.82.

### Example 40

### 4-(4-aminobutyrylamino)-1-methyl-1H-pyrrole-2-carboxylic acid (2-dimethyl-aminoethyl)amide

{3-[5-(2-Dimethylaminoethylcarbamoyl)-1-methyl-1H-pyrrol-3-ylcarbamoyl]propyl}carbamic acid tert-butyl ester (862 mg, 2.18 mmol) in 1N HCl aqueous solution in ethanol (10 mL) was heated at 60° C. for 30 min. The mixture was cooled in an ice-bath and neutralized with aqueous ammonia solution and then evaporated under reduced pressure to dryness. The solid residue was recrystallized from ethanol to give 353 mg (55%); mp 108-109° C.; ¹H NMR (DMSO-d₆) δ 1.84 (2H, t, J=7.32 Hz, CH₂), 2.34 (2H, t, J=7.28 Hz, CH₂), 2.73 (6H, s, 2×NMe), 2.79 (4H, m, 2×CH₂), 3.11 (2H, m, CH₂), 3.78 (3H, s, Me), 6.80 (1H, s, ArH), 7.14 (1H, s, ArH), 7.70 (2H, exchangeable, NH₂), 8.17 (1H, exchangeable, NH), 9.87 (1H, exchangeable, NH). Anal. Calcd. for C₁₅H₂₅N₅O·3HCl·3H₂O: C, 39.61; H, 7.53; N, 15.40. Found. C, 39.79; H, 7.64; N, 15.59.

### Example 41

### 9-(4-{2-Bis-(2-chloroethyl)amino}ethoxy)phenylamino)-5-methylacridine-4-carboxylic acid {3-[5-(2-dimethylaminoethylcarbamoyl)-1-methyl-1H-pyrrol-3-yl-carbamoyl]propyl}amide (59)

A mixture of 5-methyl-9-oxoacridan-4-carboxylic acid (126.6 mg, 0.5 mmol) was treated with thionyl chloride (2 mL) containing 2 drops of DMF was heated at 60° C. for 30 min. The reaction mixture was evaporated in vacuo to dryness and the residue was coevaporated several times with dry benzene. The residue was dissolved in CHCl₃ (20 mL) and then added into a solution of 4-(4-aminobutyrylamino)-1-methyl-1H-pyrrole-2-carboxylic acid (2-dimethylaminoethyl)amide (146 mg, 0.5 mmol) in DMF (10 mL) containing triethylamine (1.0 g, 10 mmol) and was stirred in an ice-bath for 4 h. When all starting materials were consumed, a solution of bis-(2-chloroethyl)-(3-aminobenzyl)amine (freshly prepared from bis-(2-chloroethyl)-(3-nitrobenzyl)amine [74, 157 mg, 0.5 mmol, SnCl₂ (338 mg, 1.5 mmol) in conc HCl] in CHCl₃ (20 mL). The mixture was acidified with 2.5 M HCl/EtOAc and then stirred at room temperature overnight. The reaction mixture was then evaporated in vacuo to dryness and residue was chromatographed on a silica gel column (2×30 cm) using CHCl₃/MeOH (5:1 v/v) as the eluant. The product 59 was obtained as orange solid, 222 mg (58%); mp 214-215° C.; ¹H NMR (DMSO-d₆) δ 1.97 (2H, m, CH₂), 2.42 (2H, s, CH₂), 2.73 (3H, s, Me), 2.79 (6H, m, 2×NMe), 3.20 (2H, s, CH₂), 3.30 (4H, brs, 2×NCH₂), 3.53 (4H, m, 2×CH₂), 3.75 (3H, s, Me), 3.90 (4H, brs, 2×CH₂Cl), 4.20 (2H, br, ArCH₂), 6.73-6.75 (1H, m, ArH), 7.08-7.10 (1H, m, ArH), 7.40 (1H, m, ArH), 7.51-7.57 (4H, m, ArH), 7.94 (1H, m, ArH), 8.00 (1H, brs, NH), 8.22 (1H, m, ArH), 8.46 (1H, s, NH), 8.62 (1H, m, ArH), 8.80 (1H, s, NH), 8.85 (1H, m, ArH), 9.78 (1H, s, NH). Anal. Calcd. for C₄₀H₄₉Cl₂N₈O₂·4HCl·3H₂O: C, 50.86; H, 6.30; N, 11.86. Found. C, 50.77; H, 6.48; N, 11.98.

### Example 42

### Cytotoxicity Assays

The effects of the compounds on cell growth were determined in all human tumor cells (i.e. T-cell acute lymphocytic leukemia CCRF-CEM), in a 72 h incubation, by XTT-tetrazolium assay, as described by Scudiero et al., Cancer Res. 1988, 48, 4827-4833. After the addition of phenazine methosulfate-XTT solution at 37° C. for 6 h, absorbance at 450 and 630 nm was detected on a microplate reader (EL 340; Bio-Tek Instruments Inc., Winooski, Vt.). Six to seven concentrations of each compound were used. The IC₅₀ and dose-effect relationships of the compounds for antitumor activity were calculated by a median-effect plot (see, e.g., Chou et al. Adv. Enzyme Regul. 1984, 22, 27-55) using a computer program on an IBM-PC workstation (see, e.g., Chou et al. Dose-Effect Analysis with Microcomputers: Quantitation of ED₅₀, LD₅₀, Synergism, Antagonism, Low-Dose Risk, Receptor-Ligands Binding and Enzyme Kinetics; Biosoft: Cambridge, U.K., 1987).

### Example 43

### In Vivo Assays

Athymic nude mice bearing the nu/nu gene were used for human breast tumor MX-1 xenograft. Outbred Swiss-background mice were obtained from Charies River Breeding Laboratories. Male mice 8 weeks old or older weighing 22 g or more were used for most experiments. Drug was administrated via the tail vein by i.v. injection. Tumor volumes were assessed by measuring length×width×height (or width) by using caliper. Vehicle used was 20 μl DMSO in 180 μl saline. All animal studies were conducted in accordance with the guidelines of the National Institutes of Health Guide for the Care and Use of Animals and the protocol approved by the Memorial Sloan-Kettering Cancer Center's Institutional Animal Care and Use Committee.

The cytotoxicity of selected 9-anilinoacridines against human lymphoblastic leukemic cells (CCRF-CEM) is provided in Tables 1, 2, and 3. The IC₅₀ values for compounds 37-43, 45-49, 50-53, and 54-58 were less than the IC₅₀ value for the reference compound, 3-(9-acridinylamino)-5-hydroxymethylaniline (AHMA, see e.g., Su, T.-L., et al. J. Med. Chem. 1995, 38, 3226-3235).

Data summarizing the growth inhibition of human lymphoblastic leukemic cells (CCRF-CEM) and its drug-resistant sublines (resistant to vinblastine and taxol, CCRF-CEM/VBL and CCRF-CEM/taxol, respectively) by compound 37 is shown in Table 4. Compound 37 was determined to be about 79-fold more cytotoxic than AHMA and does not develop cross-resistance to vinblastine or taxol. While not wishing to be bound by theory, this data suggests that compound 37 may not be an desirable substrate of MDR p-glycoprotein or mutated tublin. In addition, no alteration of the potency was found when the drug was washed away after incubation for 24 h. Whereas, under similar experimental conditions used to determine cytotoxicity of compound 37, the cytotoxicity of AHMA reduced potency about 12-fold. While not wishing to be bound by theory, this data suggests covalent binding to the target (DNA or Topo II) by 37. Comparison of Topo II-mediated relaxation of pRYG-DNA induced by VP-16, compound 37 and AHMA-tert-butylcarbamate revealed that these agents inhibited DNA relaxation. However, compound 37 binds relatively tightly to DNA. While not wishing to be bound by theory, this observation suggests that 37 may cross-link to DNA.

Table 5 summarizes data related to the therapeutic effects and toxicity of compound 37 (1-2 mg/kg (Q3DX7) or (3 mg/kg (Q4D×5); intravenous injection) on nude mice (n=3) bearing human breast tumor, MX-1, xenografts. Treatment with compound 37 resulted in 66.7% (2/3) of mice becoming tumor-free, whereas none (0/3) of the controlled-treated animals became tumor-free.

Table 6 summarizes data related to the therapeutic effects and toxicity of compound 37 in nude mice bearing human T-cell leukemic lymphoma CCRF-CEM xenografts. About 70% of average tumor sized was reduced at the dose of 2 mg/kg (Q3D×5).

TABLE 1
The in vitro cytotoxicity of 9-anilinoacridines linked to N-mustard (Type 1, N-
mustard moiety on anilino ring) against human lymphoblastic leukemic cells (CCRF-CEM).

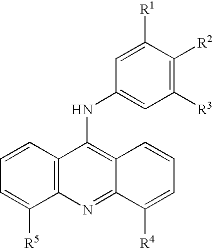

| | | | | | | IC50 |
|---|---|---|---|---|---|---|
| Compd | R1 | R2 | R3 | R4 | R5 | (μM) |
| AHMA | | | | | | 0.753 |
| 3 | | | | | | |
| 37 | O(CH2)2N(CH2CH2Cl)2 | | CH2OH | | | 0.0070 |
| 38 | O(CH2)2N(CH2CH2Cl)2 | | | | | 0.095 |
| 39 | O(CH2)4N(CH2CH2Cl)2 | | | | | 0.4555 |
| 40 | | O(CH2)2N(CH2CH2Cl)2 | | | | 0.0200 |
| 41 | | O(CH2)4N(CH2CH2Cl)2 | | | | 0.0610 |
| 42 | O(CH2)2N(CH2CH2Cl)2 | | | CONH(CH2)2NMe2 | Me | 0.0030 |
| 43 | O(CH2)4N(CH2CH2Cl)2 | | | CONH(CH2)2NMe2 | Me | 0.0235 |
| 44 | | O(CH2)2N(CH2CH2Cl)2 | | CONH(CH2)2NMe2 | Me | 0.7730 |
| 45 | | O(CH2)4N(CH2CH2Cl)2 | | CONH(CH2)2NMe2 | Me | 0.0230 |
| 46 | CH2N(CH2CH2Cl)2 | | | | | 0.0074 |
| 47 | | CH2N(CH2CH2Cl)2 | | | | 0.0300 |
| 48 | CH2N(CH2CH2Cl)2 | | | CONH(CH2)2NMe2 | Me | 0.0017 |
| 49 | | CH2N(CH2CH2Cl)2 | | CONH(CH2)2NMe2 | Me | 0.0081 |

TABLE 2
The in vitro cytotocivity of 9-anilinoacridines linked to N-mustard (Type 2, N-
mustard moiety on acridine ring) against human lymphoblastic leukemic cells
(CCRF-CEM).

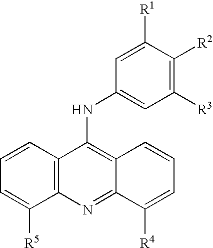

| Compd | R1 | R2 | R3 | R4 | R5 | IC50 (μM) |
|---|---|---|---|---|---|---|
| 50 | NH2 | | CH2OH | O(CH2)2N(CH2CH2Cl)2 | | 0.0072 |
| 51 | NHCOOEt | | CH2OH | O(CH2)2N(CH2CH2Cl)2 | | 0.0260 |
| 52 | NH2 | | CH2OH | O(CH2)4N(CH2CH2Cl)2 | | 0.0046 |
| 53 | OH | | CH2OH | O(CH2)2N(CH2CH2Cl)2 | | 0.0061 |

TABLE 3
The in vitro cytotocivity of 9-anilinoacridines linked to N-mustard (Type 3, N-
mustard moiety on anilino and acridine rings) against human lymphoblastic leukemic cells
(CCRF-CEM).

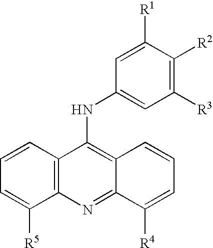

| | | | | | | IC50 |
|---|---|---|---|---|---|---|
| Compd | R1 | R2 | R3 | R4 | R5 | (μM) |
| 54 | O(CH2)2N(CH2CH2Cl)2 | | | O(CH2)2N(CH2CH2Cl)2 | | 0.0186 |
| 55 | O(CH2)4N(CH2CH2Cl)2 | | | O(CH2)2N(CH2CH2Cl)2 | | 0.0060 |
| 56 | | O(CH2)2N(CH2CH2Cl)2 | | O(CH2)2N(CH2CH2Cl)2 | | 0.0173 |
| 57 | | O(CH2)2N(CH2CH2Cl)2 | | O(CH2)4N(CH2CH2Cl)2 | | 0.00167 |
| 58 | | O(CH2)4N(CH2CH2Cl)2 | | O(CH2)2N(CH2CH2Cl)2 | | 0.0250 |

TABLE 4
The cytotoxicity of (3-(Acridin-9-ylamino)-5-{2-[bis(2-
chloroethyl)amino]ethoxy}phenyl)methanol (37) against human tumor cell growth in vitro.a

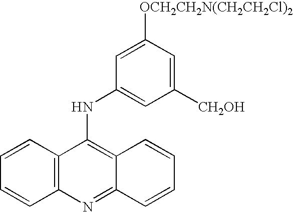

| | IC50 (μM) | | | | |
|---|---|---|---|---|---|
| | Lymphoblastic leukemic | | | Solid tumors | |
| Compound | CCRF-CEM | CCRF-CEM/VBL | CCRF-CEM/Taxol | A549 | HCT-116 |
| 37 | 0.0095 ± 0.0025 | 0.0075 [0.53×] | 0.0340 ± 0.013 [1.3×] | 0.0056 ± 0.0012 | 0.0055 ± 0.0010 |
| | | | | [0.0050]b | |
| AHMA (3) | 0.753 ± 0.378 | 1.60 [2.1×] | 0.600 ± 0.074 [0.8×] | 0.0470 [0.55]b | ND |
| Taxol | 0.0015 ± 0.0005 | 1.62 [1080×] | 0.143 ± 0.007 [95.3×] | 0.0019 [0.390]b | 0.0013 |
| Vinblastine | 0.0012 | 0.540 [450×] | 0.029 [24.2×] | 0.0081 [0.095]b | 0.0014 |

aXTT assays were used for leukemia cells and SRB assays were used for solid tumor cells. Incubation was 72 hours, as described previously (Chou et al, Proc. Natl. Acad. Sci. USA, 2001, 98, 8113-8118). Numbers in the bracket are folds of resistance of the resistant cells when compared with the IC50's of the CCRF-CEM parent cells;
bIncubation for 3 hours, washed, and then incubated for a total of 72 hours. Washing did not affect BO-742 (37) efficacy, whereas efficacy for AHMA, taxol and vinblastine were reduced due to washing;
c ND: not determined

TABLE 5
Therapeutic effects and toxicity of (3-(acridin-9-ylamino)-5-{2-[bis(2-
chloroethyl)amino]ethoxy}phenyl)methanol (37) in nude mice bearing human mammary carcinoma
(MX-1) xenograftsa

| | | | Average Body Weight bChange | | | | | | Average Tumor Size | | | | | |
|---|---|---|---|---|---|---|---|---|---|---|---|---|---|---|
| | Dose | | (gm) | | | | | | (T/C) | | | | Tumor | Toxicity |
| | mg/kg | Schedule | D11 | D14 | D17 | D20 | D23 | D26 | D17 | D20 | D23 | D26 | free | (death) |
| Control | | | 29.2 | +1.6 | +1.5 | +1.5 | +1.9 | —c | 1.0 | 1.0 | 1.0 | 1.0 | 0/3 | 0/3 |
| 37 | 1-2 | Q3Dx7 | 28.3 | +1.2 | −1.4 | −3.0 | −3.0 | −3.9 | 0.37 | 0.17 | 0.14 | ND | 2/3 | 0/3 |
| | | FIG. | | | | | | | | | | | (D32.32) | |
| | | 1A&B | | | | | | | | | | | | |
| | 3 | Q4Dx5 | 30.1 | −3.5 | −3.3 | −4.7 | −7.2 | −8.8 | 0.26 | 0.03 | 0.01 | ND | 2/3 | 3/3 |
| | | FIG. | | | (D15) | (D19) | | (D27) | (D15) | (D19) | | | (D27, 28) | (D27, 28, 29) |
| | | 2A&b | | | | | | | | | | | | |

aAMX-1 tissue 50 mg was implanted S.C. on Day 0. Treatment (i.v. injection) began on D11 when tumor size were 80~120 mm3.
bBody weight = Total body weight − Tumor weight.
cAnimal were sacrificed when there was excessive tumor burden (e.g. tumor size > 3500 mm3).

TABLE 6
Therapeutic effects and toxicity of (3-(acridin-9-ylamino)-5-{2-[bis(2-
chloroethyl)amino]ethoxy}phenyl)methanol (37) in nude mice bearing human T-cell lymphoblastic
leukemic lymphoma xenografts (CCRF-CEM)a

| | | | Average Body | | | | | | | | | | | |
|---|---|---|---|---|---|---|---|---|---|---|---|---|---|---|
| | | | Weightb Change | | | | | | Average Tumor Size | | | | | |
| | Dose | | (gm) | | | | | | (T/C) | | | | Tumor | Toxicity |
| | (mg/kg) | Schedule | D10 | D13 | D16 | D19 | D22 | D25 | D16 | D19 | D22 | D25 | free | (death) |
| Control | | | 26.3 | −0.7 | −0.8 | +1.6 | +2.1 | +2.3 | 1.0 | 1.0 | 1.0 | 1.0 | 0/3 | 0/3 |
| 37 | 2 | Q3Dx5 | 29.4 | −1.1 | −4.2 | −3.3 | −5.9 | −6.8 | 0.47 | 0.34 | 0.33 | 0.34 | 0/3 | 0/3 |
| | | FIG. 4A&B | | | | | | | | | | | | |

aCCRF-CEM tissue 50 mg was implanted S.C. on D0. Treatment (i.v. injection) began on D10 when tumor size were 120 mm3.
bBody weight = Total body weight − Tumor weight.

A number of embodiments of the invention have been described. Nevertheless, it will be understood that various modifications may be made without departing from the spirit and scope of the invention. Accordingly, other embodiments are within the scope of the following claims.

What is claimed is:

1. A compound having formula (I): 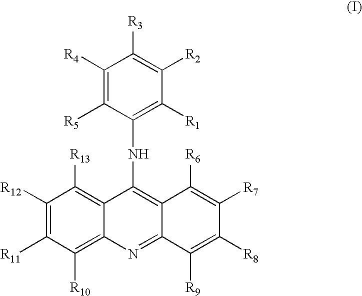
wherein,
each of R₁, R₂, R₃, R₄, R₅, R₆, R₇, R₈, R₉, R₁₀, R₁₁, R₁₂, and R₁₃ is, independently, hydrogen, halo, nitro, hydroxyl, C₁-C₆ alkyl, C₁-C₆ alkoxy, C₁-C₆ hydroxyalkyl, CONHR^{a}, NR^{b}R^{c}, CONH(CH₂)ₘNR^{b}R^{c}, L-N(CH₂CH₂Cl)₂, or a DNA minor groove binder;
L is (CH₂)ₚ or O(CH₂)_{q};
m is 1, 2, 3, or 4;
p is 0, 1, 2, 3, or 4;
q is 1, 2, 3, 4, 5, 6, 7, or 8;
in which, R^{a} is C₁-C₆ alkyl; each of R^{b} and R^{c} is, independently, hydrogen, C₁-C₆ alkyl, COR^{d}, or COOR^{d}; R^{d} is C₁-C₆ alkyl, C₆-C₁₀ aryl, or C₇-C₁₂ aralkyl; and provided that at least one of R₁, R₂, R₃, R₄, R₅, R₆, R₇, R₈, R₉, R₁₀, R₁₁, R₁₂, and R₁₃ is L-N(CH₂CH₂Cl)₂, or a salt thereof.

2. The compound of claim 1, wherein L is (CH₂)ₚ.

3. The compound of claim 2, wherein p is 0 or 1.

4. The compound of claim 1, wherein L is O(CH₂)_{q}.

5. The compound of claim 4, wherein q is 2 or 4.

6. The compound of claim 1, wherein one of R₁, R₂, R₃, R₄, or R₅ is L-N(CH₂CH₂Cl)₂.

7. The compound of claim 6, wherein R₂ or R₃ is L-N(CH₂CH₂Cl)₂.

8. The compound of claim 7, wherein R₂ is L-N(CH₂CH₂Cl)₂.

9. The compound of claim 8, wherein L is (CH₂)ₚ.

10. The compound of claim 9, wherein p is 0 or 1.

11. The compound of claim 8, wherein L is —O(CH₂)_{q}—.

12. The compound of claim 11, wherein q is 2 or 4.

13. The compound of claim 8, wherein each of R₁, R₃, R₄, and R₅ is, independently, hydrogen, C₁-C₆ alkyl, C₁-C₆ alkoxy, or C₁-C₆ hydroxyalkyl.

14. The compound of claim 13, wherein R₄ is C₁-C₆ hydroxyalkyl.

15. The compound of claim 14, wherein R₄ is CH₂OH.

16. The compound of claim 13, wherein each of R₁, R₃, R₄, and R₅ is hydrogen.

17. The compound of claim 7, wherein R₃ is L-N(CH₂CH₂Cl)₂.

18. The compound of claim 17, wherein L is (CH₂)ₚ.

19. The compound of claim 18, wherein p is 0 or 1.

20. The compound of claim 17, wherein L is —O(CH₂)_{q}—.

21. The compound of claim 20, wherein q is 2 or 4.

22. The compound of claim 17, wherein each of R₁, R₂, R₄, and R₅ is, independently, hydrogen, C₁-C₆ alkyl, C₁-C₆ alkoxy, or C₁-C₆ hydroxyalkyl.

23. The compound of claim 21, wherein each of R₁, R₂, R₄, and R₅ is hydrogen.

24. The compound of claim 6, wherein each of R₆, R₇, R₈, R₉, R₁₀, R₁₁, R₁₂, and R₁₃ is, independently, hydrogen, halo, nitro, C₁-C₆ alkyl, C₁-C₆ alkoxy, CONHR^{a}, CONH(CH₂)ₘNR^{b}R^{c}, L-N(CH₂CH₂Cl)₂, or a DNA minor groove binder.

25. The compound of claim 24, wherein each of R₆, R₇, R₈, R₉, R₁₀, R₁₁, R₁₂, and R₁₃ is, independently, hydrogen, C₁-C₆ alkyl, CONH(CH₂)ₘNR^{b}R^{c}, L-N(CH₂CH₂Cl)₂, or a DNA minor groove binder.

26. The compound of claim 25, wherein one of R₉ and R₁₀ is CONH(CH₂)ₘNR^{b}R^{c}, L-N(CH₂CH₂Cl)₂, or a DNA minor groove binder, and the other is C₁-C₆ alkyl or hydrogen.

27. The compound of claim 26, wherein one of R₉ and R₁₀ is CONH(CH₂)ₘNR^{b}R^{c} and the other is C₁-C₆ alkyl or hydrogen.

28. The compound of claim 27, wherein one of R₉ and R₁₀ is CONH(CH₂)₂N(CH₃)₂ and the other is CH₃ or hydrogen.

29. The compound of claim 26, wherein one of R₉ and R₁₀ is L-N(CH₂CH₂Cl)₂ and the other is C₁-C₆ alkyl or hydrogen.

30. The compound of claim 29, wherein one of R₉ and R₁₀ is N(CH₂CH₂Cl)₂ or CH₂N(CH₂CH₂Cl)₂ and the other is CH₃ or hydrogen.

31. The compound of claim 29, wherein one of R₉ and R₁₀ is O(CH₂)₂N(CH₂CH₂Cl)₂ or O(CH₂)₄N(CH₂CH₂Cl)₂ and the other is CH₃ or hydrogen.

32. The compound of claim 26, wherein one of R₉ and R₁₀ is a DNA minor groove binder and the other is C₁-C₆ alkyl or hydrogen.

33. The compound of claim 32, wherein one of R₉ and R₁₀ is CONH(CH₂)ᵣ-J-W—(CH₂)ₜR^{e} and the other is CH₃ or hydrogen; wherein r is 1, 2, 3, 4, or 5; t is 1, 2, 3, or 4, 5, or 6; J is —CONH— or —NHCO—; W is: 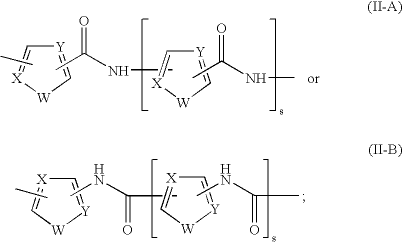
in which s is 0, 1, 2, 3, or 4; each of X and Y is, independently, N or CR^{f} and W is NR^{g}, O, or S; R^{e} is NR^{b}R^{c}, NHCHO, or NHC(═NH)NH₂; each of R^{b} and R^{c} is, independently, hydrogen, C₁-C₆ alkyl, COR^{d}, or COOR^{d}; and each of R^{f} and R^{g} is, independently, hydrogen or C₁-C₆ alkyl.

34. The compound of claim 33, wherein s is 0, each of X and Y is CH, and W is NCH₃.

35. The compound of claim 34, wherein one of R₉ and R₁₀ is:

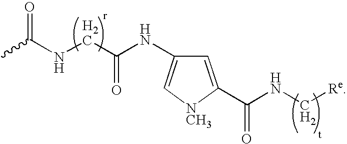

36. The compound of claim 35, wherein r and t are both 3, and R^{e} is N(CH₃)₂, NHCHO, or NHC(═NH)NH₂.

37. The compound of claim 34, wherein one of R₉ and R₁₀ is:

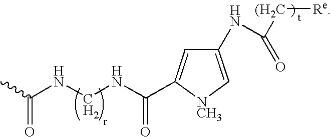

38. The compound of claim 36, wherein r and t are both 3, and R^{e} is N(CH₃)₂, NHCHO, or NHC(═NH)NH₂.

39. The compound of claim 24, wherein each of R₆, R₇, R₈, R₉, R₁₀, R₁₁, R₁₂, and R₁₃ is hydrogen.

40. The compound of claim 1, wherein one of R₆, R₇, R₈, R₉, R₁₀, R₁₁, R₁₂, and R₁₃ is L-N(CH₂CH₂Cl)₂.

41. The compound of claim 40, wherein R₉ is L-N(CH₂CH₂Cl)₂.

42. The compound of claim 41, wherein L is (CH₂)ₚ.

43. The compound of claim 42, wherein p is 0 or 1.

44. The compound of claim 41, wherein L is —O(CH₂)_{q}—.

45. The compound of claim 44, wherein q is 2 or 4.

46. The compound of claim 41, wherein each of R₆, R₇, R₈, R₁₀, R₁₁, R₁₂, and R₁₃ is, independently, hydrogen, halo, nitro, hydroxyl, C₁-C₆ alkyl, or C₁-C₆ alkoxy.

47. The compound of claim 40, wherein each of R₁, R₂, R₃, R₄, or R₅ is, independently, hydrogen, hydroxyl, C₁-C₆ alkyl, C₁-C₆ alkoxy, C₁-C₆ hydroxyalkyl, or NR^{b}R^{c}.

48. The compound of claim 47, wherein R₂ is hydroxyl or NR^{b}R^{c} and R₄ is C₁-C₆ hydroxyalkyl.

49. The compound of claim 48, wherein R₂ is NH₂ or NHCOOCH₂CH₃.

50. The compound of claim 48, wherein R₄ is CH₂OH.

51. The compound of claim 1, wherein the compound is:

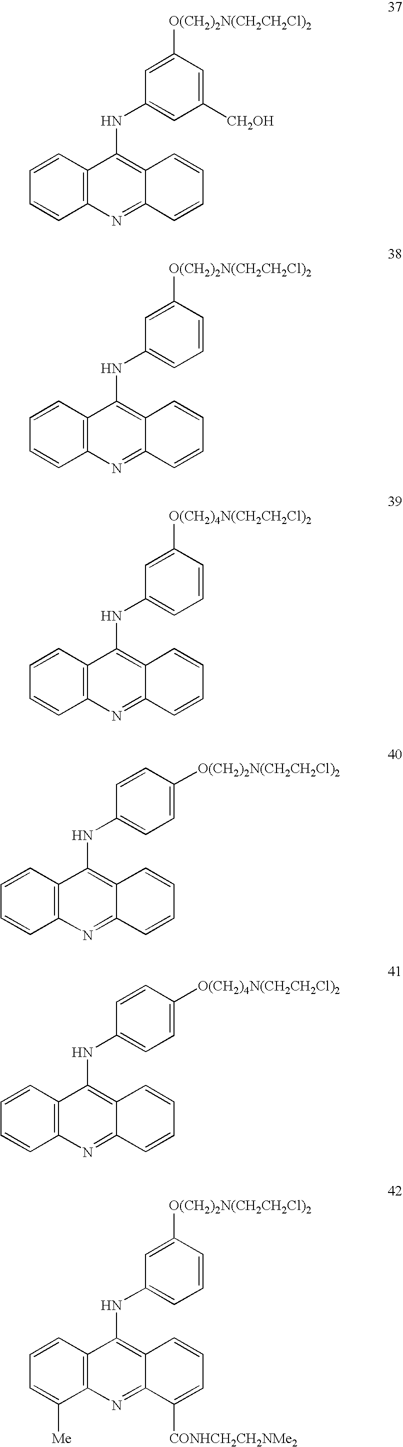

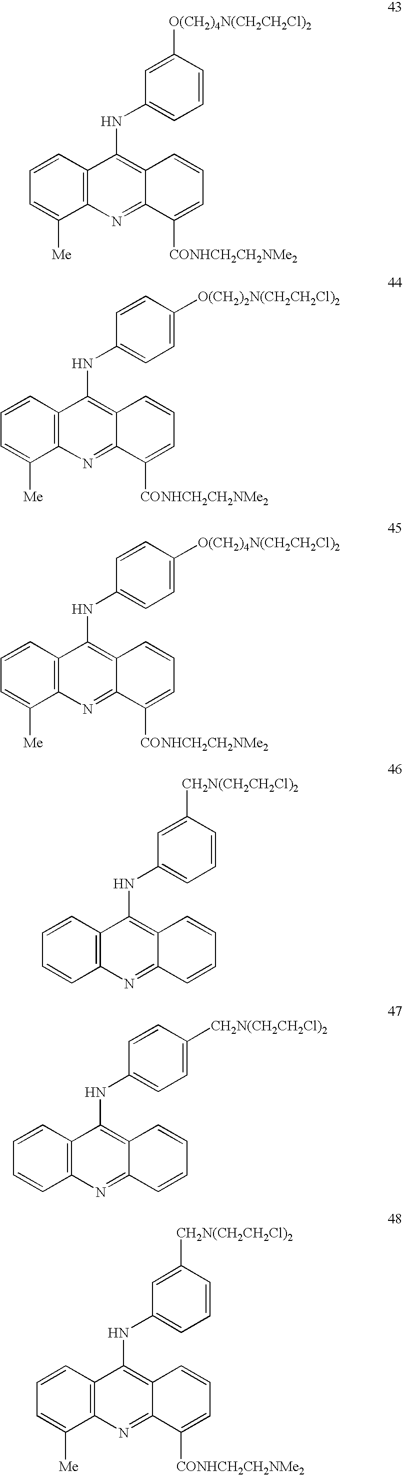

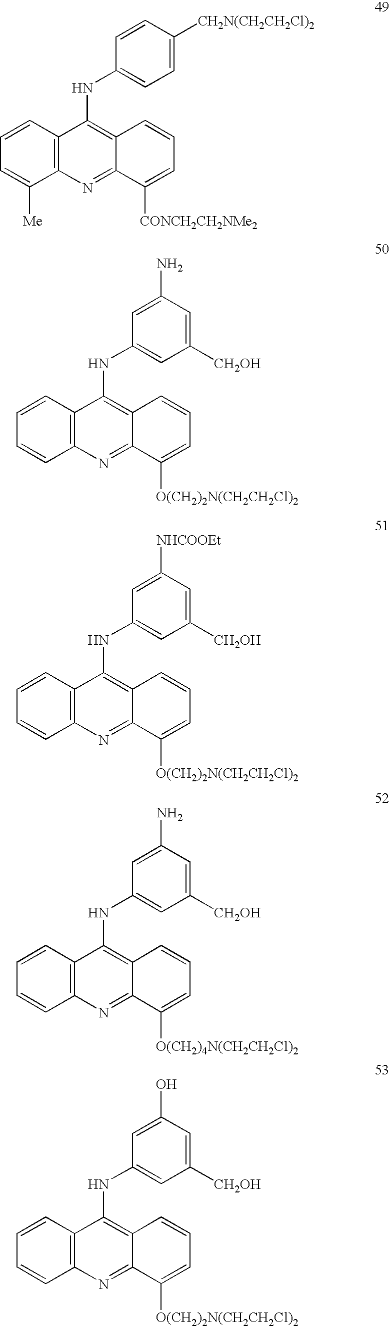

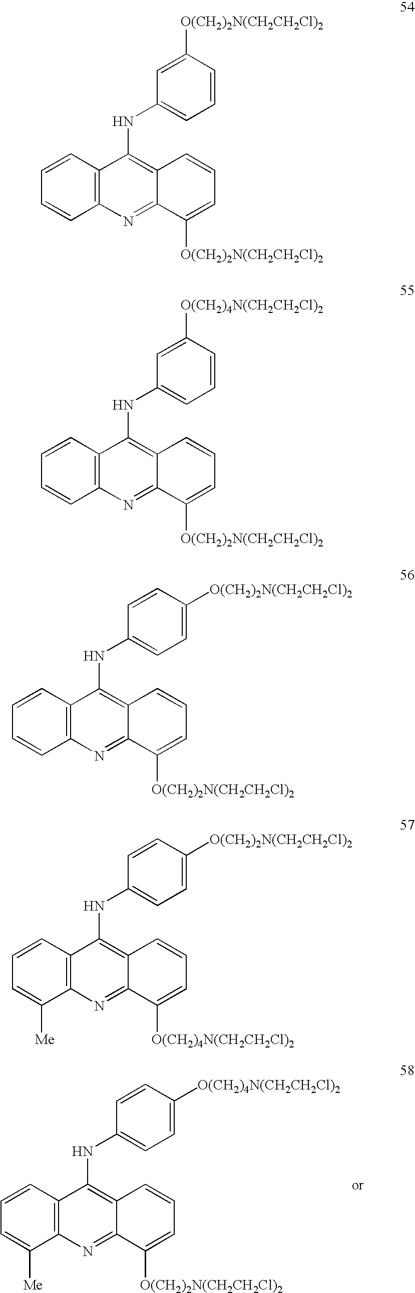

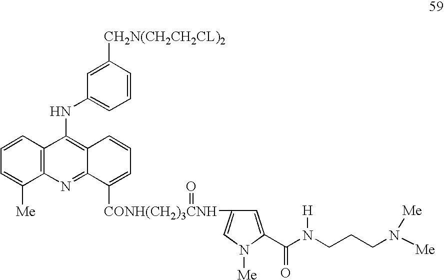

52. A pharmaceutical composition comprising a compound of formula (I) and a pharmaceutically acceptable carrier: 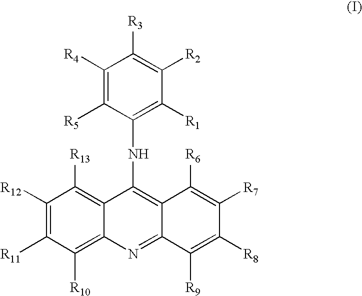
wherein,
each of R₁, R₂, R₃, R₄, R₅, R₆, R₇, R₈, R₉, R₁₀, R₁₁, R₁₂, and R₁₃ is, independently, hydrogen, halo, nitro, hydroxyl, C₁-C₆ alkyl, C₁-C₆ alkoxy, C₁-C₆ hydroxyalkyl, CONHR^{a}, NR^{b}R^{c}, CONH(CH₂)ₘNR^{b}R^{c}, L-N(CH₂CH₂Cl)₂, or a DNA minor groove binder;
L is (CH₂)ₚ or O(CH₂)_{q};
m is 1, 2, 3, or 4;
p is 0, 1, 2, 3, or 4;
q is 1, 2, 3, 4, 5, 6, 7, or 8;
in which, R^{a} is C₁-C₆ alkyl; each of R^{b} and R^{c} is, independently, hydrogen, C₁-C₆ alkyl, COR^{d}, or COOR^{d}; R^{d} is C₁-C₆ alkyl, C₆-C₁₀ aryl, or C₇-C₁₂ aralkyl; provided that at least one of R₁, R₂, R₃, R₄, R₅, R₆, R₇, R₈, R₉, R₁₀, R₁₁, R₁₂, and R₁₃ is L-N(CH₂CH₂Cl)₂; or a pharmaceutically acceptable salt thereof.

53. A method of treating cancer, the method comprising administering to a subject in need thereof an effective amount of a compound of formula (I): 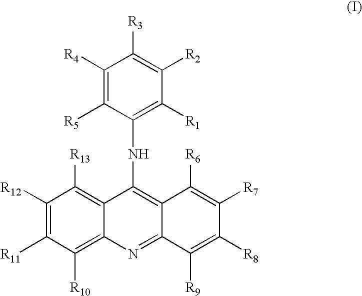
wherein,
each of R₁, R₂, R₃, R₄, R₅, R₆, R₇, R₈, R₉, R₁₀, R₁₁, R₁₂, and R₁₃ is, independently, hydrogen, halo, nitro, hydroxyl, C₁-C₆ alkyl, C₁-C₆ alkoxy, C₁-C₆ hydroxyalkyl, CONHR^{a}, NR^{b}R^{c}, CONH(CH₂)ₘNR^{b}R^{c}, L-N(CH₂CH₂Cl)₂, or a DNA minor groove binder;
L is (CH₂)ₚ or O(CH₂)_{q};
m is 1, 2, 3, or 4;
p is 0, 1, 2, 3, or 4;
q is 1, 2, 3, 4, 5, 6, 7, or 8;
in which, R^{a} is C₁-C₆ alkyl; each of R^{b} and R^{c} is, independently, hydrogen, C₁-C₆ alkyl, COR^{d}, or COOR^{d}; R^{d} is C₁-C₆ alkyl, C₆-C₁₀ aryl, or C₇-C₁₂ aralkyl; and provided that at least one of R₁, R₂, R₃, R₄, R₅, R₆, R₇, R₈, R₉, R₁₀, R₁₁, R₁₂, and R₁₃ is L-N(CH₂CH₂Cl)₂; or a pharmaceutically acceptable salt thereof.
